SEQUENCE LISTING (6 sequences)

SEQ ID NO: 1 - DNA, 23 bp, Artificial Sequence
  Target-1 oligonucleotide (miRNA-20a)
taaagtgcttatagtgcaggtag

SEQ ID NO: 2 - DNA, 23 bp, Artificial sequence
  Target-2 oligonucleotide (miRNA-20b)
caaagtgctcatagtgcaggtag

SEQ ID NO: 3 - DNA, 27 bp, Artificial Sequence
  Target-3 oligonucleotide (longer)
cttaaagtgcttatagtgcaggtagag

SEQ ID NO: 4 - DNA, 19 bp, Artificial Sequence
  Target-3 oligonucleotide (shorter)
aagtgcttatagtgcaggt

SEQ ID NO: 5 - DNA, 36 bp, Artificial Sequence
  Probe-C1/F1 oligonucleotide
gtgctgaccgtagtaccggtcagcacctacctgcac

SEQ ID NO: 6 - DNA, 39 bp, Artificial Sequence
  Probe-C2/F2 oligonucleotide
tataagcactttagcgtgcagccatactaggctgcacgc

(19) United States
(12) Patent Application Publication
(10) Pub. No.: US 2023/0212652 A1
(43) Pub. Date: Jul. 6, 2023

(54) Luminescence hybridisation assay method

(71) Applicant: Iptomics Oy, Turku (FI)

(72) Inventor: Tero Soukka, Turku (FI)

(21) Appl. No.: 17/793,960

(22) PCT Filed: Jan. 20, 2021
(86) PCT No.: PCT/FI2021/050031

§ 371 Date: Jul. 20, 2022

(30) Foreign Application Priority Data
Jan. 20, 2020 (FI) 20207013

(51) Int. Cl. C12Q 1/6818

(52) U.S. Cl. CPC C12Q 1/6818

(57) ABSTRACT

This invention relates to a bioassay method for detecting and/or quantitating a short single-stranded nucleic acid analyte employing a binary probe system, where at least one of the two discrete oligonucleotide probe parts of the binary probe has partially double-stranded (self-complementary) stem-loop structure at one terminus and single-stranded overhang sequence region at the other terminus, where the single-stranded terminal regions of both discrete parts of the binary probe hybridize to adjacent complementary regions in the sequence of the nucleic acid analyte molecule, and at least one discrete part of the binary probe comprising a stem-loop structure and single-stranded overhang sequence region hybridizes to terminal region in the sequence of the nucleic acid analyte molecule forming a nick structure. The binary probe system employed in the bioassay method is based on a luminescent reporter technology, either lanthanide chelate complementation or resonance energy transfer with lanthanide label as a donor. Thereby the method allows detection and/or quantitation of the short nucleic acid analyte molecule by time-resolved fluorometry.

### FIELD OF THE INVENTION

This invention relates to a luminescence hybridization bioassay method to detect and/or quantitate a short nucleic acid analyte molecule.

### BACKGROUND OF THE INVENTION

The publications and other materials used herein to illuminate the background of the invention, and in particular, cases to provide additional details respecting the practice, are incorporated by reference.

A number of assays based on bioaffinity binding reactions or enzymatically catalyzed reactions have been developed to analyze concentration of biologically important compounds such as nucleic acids from, or their activity or their biological effect or its modulation induced by, various biological or clinical samples, samples in environmental studies or from industrial processes. Some of these assays rely on specific bioaffinity recognition reactions, where e.g. natural biological binding components or artificially produced biospecific binding compounds are used to recognize the target compound. Example of such artificially produced biospecific binding compounds are synthetic oligonucleotide probes that are complementary in their sequence to the sequence fragment of the nucleic acid analyte. In addition to the direct measurement of the concentration of the target compound recognized by the biospecific binding reaction, the assays can indirectly measure the activity or modulation of the activity of other compounds present in sample or added into reaction (e.g. biologically active enzymes, chemical compounds with activity on biological molecules, enzyme substrates, enzyme activators, enzyme inhibitors, enzyme modulating compounds) and so on.

Generally bioaffinity binding assays rely on a label, i.e. a reporter molecule, or a combination of multiple labels responsible for signal generation to e.g. visualise or quantitate the amount of complexes formed between the biospecific binding compound or compounds and target compound. A label is a compound attached directly or indirectly to the biospecific binding compounds. An example of the labeled biospecific binding compounds is an oligonucleotide probe that comprises a label moiety covalently attached to the synthetic oligonucleotide either via terminal backbone modification or via modified nucleobase. In heterogeneous assays a separation step (separations like precipitation and centrifugation, filtration, solvent extraction, gel filtration or other chromatographic technique or affinity collection to e.g. plastic surfaces such as coated assay tubes, slides or microparticles,) is generally required to measure only the signal generated by the labels present in the complexes formed by the biospecific binding compound or compounds and target compound and, thus, to determine the amount of the target compound. In homogeneous assays the signal of the label or labels is modulated or generated only upon formation of the complexes between the biospecific binding compound or compounds and target compound and, thus, no separation step is needed to determine the amount of the target compound based on the measurement of the signal generated by the label or labels.

Both in heterogeneous and homogeneous assays the determination of the concentration of the target compound in the sample is generally based on measurement of the signal from series of standards with known concentration, i.e. calibrators, to which the signal from unknown sample is compared. Various bioffinity binding assay and different label and detection techniques employed have been reviewed e.g. in Principles and Practices of Immunoassay, 2nd ed., edited by C. P. Price and D. J. Newman, Nature Publishing Group, 1997; The Immunoassay Handbook, 4th ed. David Wild, Elsevier Science, 2013; Fluorescent Energy Transfer Nucleic Acid Probes: Designs and Protocols, edited by V. V. Didenko, Humana Press, 2006; and Principles and Applications of Molecular Diagnostics, edited by N. Rifai, A. R. Horvath and Carl. T. Wittwer, Elsevier Science, 2018.

### Hybridization Assays

Detection and quantitative analysis of concentration of deoxyribonucleic acid (DNA) or ribonucleic acid (RNA) target compounds in the sample by biospecific binding assays is generally based on the sequence-specific hybridization of complementary nucleic acid sequences. In the hybridization assays the labeled oligonucleotide probe or probes bind to the complementary sequence fragment or fragments present in the target nucleic acid compounds. Depending on the label and detection technique employed, the assay can be either heterogeneous or homogeneous, yet homogenous assays are often preferred also for nucleic acid analytes. The binding affinity of certain oligonucleotide probe to the complementary target nucleic acid sequence fragment at certain environmental conditions (e.g. temperature, salt concentration, and other additives such as glycerol, formamide and dimethyl sulfoxide) is generally indicated as melting temperature (Tₘ), the temperature at which one half of the maximal degree of complexation between oligonucleotide probe and target nucleic acid fragment is achieved. In addition to the environmental conditions, the melting temperature of the oligonucleotide probe is defined by the length of the complementary sequence and the base composition. The longer complementary nucleobase sequence and higher abundance of guanine and cytosine bases in the sequence results in higher melting temperature, whereas higher abundance of adenosine and thymine as well as shorter complementary sequence and presence of possible mismatches between the oligonucleotide probe and the target nucleic acid sequences result in lower melting temperature. In practice, a melting temperature clearly above the temperature of the assay conditions is required to enable efficient complex formation and stable binding of the oligonucleotide probe to the target nucleic acid sequence in order to maximize the assay sensitivity. Therefore, usually oligonucleotide probe length of at least 15 bases is necessary when natural deoxyribonucleic acid probe is used at temperatures around 30 - 40° C. Further, the longer the complementary sequence of the oligonucleotide probe is, the more likely it is that the probe can at least weakly bind also to nucleic acid sequences with only a single nucleotide difference (mismatch) compared to the fully complementary target nucleic acid. The specificity of the oligonucleotide probe hybridization to recognize single nucleotide mismatches is weakest when the mismatch is present at or close to either terminus end of the oligonucleotide probe sequence [BMC Genomics 2007: 8: 373].

Quantitative 5′-nuclease based polymerase chain reaction assay (TaqMan; Applied Biosystems, Waltham, MA) is a nucleic acid sequence detection method wherein a single-stranded self-quenched oligonucleotide probe, containing both a fluorescent moiety and a quencher moiety, is cleaved by the 5′ exonuclease action of nucleic acid polymerase upon hybridisation to the target nuelcid acid during nucleic acid amplification [Curr. Opin. Biotechnol. 1998; 9: 43-48; and Clin. Chem. Lab. Med. 1998; 36: 255-269]. This technique is, however, only applicable for detection and quantification when combined with nucleic acid amplification utilizing polymerase with 5′ exonuclease activity.

Molecular beacons are single-stranded oligonucleotide hybridization probes that form a stem-and-loop structure [Curr. Opin. Chem. Biol. 2004; 8: 547-553; Chemistry 2000; 6: 1107-1111; Nature Biotechnol. 1996; 14: 303-308]. The loop contains a nucleic acid probe sequence that is complementary to a target sequence, and the stem is formed by annealing of complementary arm sequences that are located on either side of the probe sequence. A fluorescent (dye) moiety is covalently linked to the end of one arm and a quencher (dye) moiety is covalently linked to the end of the other arm. Since the complementary stem sequences hybridize together forming a double-stranded structure, the fluorescent moiety and quencher moiety are in close proximity and the molecular beacons do not fluoresce when they are free in solution at assay temperature. However, when they hybridize to a complementary nucleic acid strand containing a target sequence they undergo a conformational change opening the double stranded stem and increasing the distance between fluorescent moiety and the quencher moiety, which enables the molecular beacon probe to fluoresce. In the absence of a complementary target sequence, the molecular beacon probe remains closed and there is no fluorescence due to intramolecular quenching. The specificity of the molecular beacon probes, however, is dependent on the single hybridization event and thus prone to mishybridization and requires careful optimization of the hybridization conditions or melting curve based analysis of the binding [Pharmacogenomics 2007; 8: 597-608] to differentiate targets with minimal sequence differences.

### Binary Probes

Most heterogeneous hybridization assays and also many of the homogeneous hybridization assay techniques are based on simultaneous recognition of the target nucleic acid molecule by a pair of two distinct oligonucleotide probes capable to hybridize simultaneously either to adjacent or more distant positions in the sequence of the target nucleic acid molecule. In heterogeneous assays this typically involves presence of labeled oligonucleotide probe and solid-phase attached capture oligonucleotide probe, whereof the former is linked to the solid-phase only in presence of the target nucleic acid. This enables a separation step to remove from the reaction the labeled probe oligonucleotides not bound to the target nucleic acid sequence [J. Clin. Lab. Anal. 1989; 3: 122-135]. In homogeneous luminescence hybridization assays commonly either self-reporting oligonucleotide probes such as molecular beacons [Nat. Biotechnol. 1998; 16: 49-53] or different binary oligonucleotide probes [Chem. Rev. 2010; 110: 4709-4723] can be utilized.

In homogeneous binary hybridization probe based assays, where fluorescence resonance energy-transfer (FRET) is utilized to detection and quantitation of the target, two separate labeled oligonucleotide probes binding to nearby or adjacent position in the target nucleic acid sequence are required: one oligonucleotide probe is labeled with a donor fluorophore and the other is labeled with an acceptor fluorophore or a quencher molecule. The donor and the acceptor, or the donor and quencher, are selected spectroscopically in such way that resonance energy transfer is possible from the donor to the acceptor or to the quencher when they are in close proximity [Can. J. Chem. (2015) 93: 389-398]. Upon binding of the two probes simultaneously to the same target nucleic acid molecule, the resulting short distance between the donor and acceptor labels enables fluorescence resonance energy-transfer between them and the presence of the target nucleic acid can detected without any separation step by decrease in the donor fluorescence and, in case of acceptor is fluorescent, also by increase of the energy-transfer sensitized acceptor remission. The specificity of target detection in binary probe hybridization assay can be further improved by adding a ligation step to join the hybridized probes covalently together at the nick site by catalytic activity of RNA or DNA ligase [Nucleic Acids Res. 2001; 29: e70]. The enzymatic ligation of nick in double stranded DNA or DNA/RNA hybrid is strictly specific to the fully complementary sequences at opposite strands around the nick site. The ligation of the hybridized probe pair combined with subsequent more stringent hybridization conditions to dissociate non-ligated probes, can partly eliminate the weak specificity of the probe hybridization, especially when the mismatch is located at terminal ends of the probe.

The dependence of the signal generation or signal modulation upon binding of two distinct probes to the same target nucleic acid molecule both in the heterogeneous and homogeneous binary probe hybridization assays renders the target nucleic acid sequence detection, in principle, more specific as mishybridisation of two separate oligonucleotide probes simultaneously to adjacent positions on the same target nucleic acid molecule is unlikely [J. Mol. Diagn. 2010; 12: 359-367]. This provides them potential advantage in specificity over the signal generation techniques that are based on hybridization of a single oligonucleotide probe such as the molecular beacon probe. The binding of two oligonucleotide probes to the same target nucleic acids, however, sets additional requirements for the target nucleic acid sequence and challenges for the assay design.

The efficient complex formation and stable binding of the two oligonucleotide probes simultaneously to the same target molecule at the assay conditions requires, that both the two distinct oligonucleotide probes have a high enough melting temperature concerning the assay conditions. This will set the minimal length of the two distinct oligonucleotide probes and also the minimal length of the target nucleic acid molecule, which can be detected by these methods, as the length of the target nucleic acid sequence must be long enough to enable simultaneously hybridization of the two distinct oligonucleotide probes to nearby or adjacent positions in the target nucleic acid sequence. This can be, however, partially facilitated by the enhanced stabilization from the base stacking effect [Nucleic Acids Res. 2006; 34: 564-574] upon hybridization of the two oligonucleotide probes to immediately adjacent positions forming a nick structure between the probes, and/or via cooperative binding of two oligonucleotide probes utilized in some binary probe approaches [Chem. Rev. 2010: 4709-4723; US. Pat. No. 8,313,903]. Generally, however, additional techniques, e.g. nucleic acid backbone analogues such as peptide nucleic acids [Science 1991; 254: 1497-500] and locked nucleic acids [J. Am. Chem. Soc. 1998; 120: 13252 - 13253], modified artificial nucleobases and minor groove binding molecules [Nucleic Acids Res. 2000; 28: 655-661], can be used and incorporated to the oligonucleotide probes to increase the stability of shorter probes.

Multiple signal generation strategies have been combined with binary probes: fluorescence resonance energy transfer based detection is widely used, but also other fluorescence based techniques with more strict proximity requirement for the label components have been demonstrated. These include e.g. lanthanide chelate complementation by light-harvesting sensitizer antenna coordination to ion carrier chelate [Angew. Chem. Int. Ed. Engl. 1990; 29: 1167-116; PCT Int. Appl. WO 2010/109065], pyrene excimer and exciplex formation, chemical cross-linking of the probes resulting in e.g. quencher removal or fluorophore activation, and aptamer based fluorophore enhancement of e.g. Malachite green and Hoechst derivative 7 [Chem. Rev. 2010; 110: 4709-4723]. Other non-fluorescent techniques include e.g. formation of deoxyriboxymes from two halves (split DNA enzymes).

Binary probe approaches can be be further enhanced by combining ligation of adjacently bound oligonucleotide probes to provide additional specificity of target recognition by ligase enzyme. For example, binary probe hybridization based gene detection utilizing ligation of two adjacently hybridized oligonucleotide probes comprising multiple stem-loop structures and Q-beta-replicase based amplification of the ligated product has been described [Proc. Natl. Acad. Sci. USA 1996; 93: 5395-5400].

### Resonance Energy Transfer

Fluorescence or Forster resonance energy transfer (FRET) is a strongly distance dependent (to inverse sixth power) non-radiative energy transfer mechanism between two properly chosen fluorescent molecules present in close proximity [Ann. Physik 1948; 2: 55-75]. Resonance energy transfer (RET) occurs at practical efficiency when a donor and an acceptor fluorophore are within Förster radius (typical values 4-7 nm) and the donor emission spectrum and the acceptor absorption spectrum overlap. The acceptor can also be a non-fluorescent. The RET is typically monitored either by measuring a decrease of donor fluorescence emission or an increase of acceptor fluorescence emission intensity (known as sensitized acceptor emission) [Biochem. Spectroscopy 1995; 246: 300-334] resulting from proximity of donor and acceptor. In case of non-fluorescent acceptor (known as quencher) a change of donor emission intensity is monitored.

Although the FRET is a widely employed and an essential technique in many applications, it has severe performance limitations with conventional fluorescent dyes when utilized in clinical samples [Clin. Chem. 1985; 31: 359-370]. FRET based biomolecular sensing is a technique capable of detecting the nearness of the FRET donor and acceptor probe pair (FRET probe pair) and is used in different assay formats for detection of various biomolecules. An individual FRET probe consists typically of a biospecific binding compound or other recognition site (with specific affinity for the target compound, i.e. analyte molecule) labeled either with single or multiple donor or acceptor moiety(ies) and the target molecule is able to direct by biomolecular binding the FRET donor and acceptor probe pair into close proximity. The short distance between the FRET donor and acceptor probes is thus provided when distinct probe molecules bind e.g. to their respective binding sites located in proximity in the target molecule. In fluorescence resonance energy transfer assays the binding of the FRET probe pair to the target molecule results in quenching of the donor emission (upon FRET from the donor to the fluorescent acceptor or non-luminescent quencher) and, in case of fluorescent acceptor, also increase in the FRET-sensitized acceptor emission.

The conventional FRET-based assays are however susceptible to i) direct excitation of the acceptor (the acceptor is weakly excited at the same wavelength where the donor is excited), ii) crosstalk of donor emission (the donor has some emission at the same wavelength where the acceptor emission is measured), iii) radiative energy transfer (less distance dependent; to inverse second power) through absorption of donor emission (photons) by acceptor fluorophores not necessarily in proximity, and iv) scattered excitation light and autofluorescence (from sample, other assay components, plastics and detection instrument itself) generating background signal. Thus, conventional fluorophores and FRET probes do not provide the optimal specificity in signal generation for the binary probe approach. Further, it is difficult to measure more than two parameters simultaneously in a multiparametric FRET-based assay due to wide spectral coverage of an individual donor-acceptor pair.

In fluorescence resonance energy-transfer based homogenous hybridization assays [Proc. Natl. Acad. Sci. USA 1988; 85: 8790-8794], where two labeled oligonucleotide probes (binary probe) hybridize to positions in close proximity on the target nucleic acid sequence and the resulting resonance-energy transfer sensitized acceptor emission is measured to quantify the presence of the target nucleic acid, problems arise from the fluorescence cross-talk between the donor and acceptor emission. The excitation of the donor will inevitably also result in direct excitation of the acceptor generating background signal, and the acceptor can also be radiatively excited by the donor emission due to spectral overlap between the donor emission and acceptor excitation. The weak affinity (low melting temperature) of short oligonucleotide probes could normally be compensated by using high concentration of the probes, but in fluorescence resonance energy transfer based assays this would result in increased background signal due to direct excitation of the acceptor and radiative sensitization of the acceptor upon reabsorption of the donor emission, and thus also reduced donor emission even in absence of the target nucleic acid. In case the fluorescent donor and acceptor combination, however, is selected to minimize the direct excitation of the acceptor by increased spectral separation of the excitation spectra of the donor and accepter, this usually results also in smaller spectral overlap between the donor emission and acceptor excitation, and thus again compromises the signal generation by reducing the energy-transfer efficiency.

Detection sensitivity of conventional fluorescence-based techniques is further limited by autofluorescence, scattered excitation light and absorbance of biological sample matrices. Many compounds and proteins present in biological fluids or serum are naturally fluorescent, and the use of conventional fluorophores leads to serious limitations of sensitivity [Clin. Chem. 1979; 25:353-361; Anal. Biochem. 1994; 218:1-13]. Another major problem when using homogeneous fluorescence techniques based on intensity measurements is the inner filter effect and the variability of the optical properties of a sample. Sample dilution has been used to correct these drawbacks, but always at the expense of analytical sensitivity. Feasibility of fluorescence resonance energy transfer in assay applications was significantly improved when fluorescent lanthanide cryptates and chelates with long-lifetime emission and large Stokes’ shift were employed as donors in the 1990’s [Clin Chem 1993; 39:1953-1959; Anal. Biochem. 1994; 218,1-13; Proc. Natl. Acad. Sci. USA 1994; 91:10024-10028; Cytokine 1998; 10:495-499; PCT Int. Appl. WO 98/15830; US. Pat. No. 5,998,146; PCT Int. Appl. WO 87/07955; Clin. Chem. 1999; 45:855-61]

### Lanthanide Complexes and Time-Resolved Fluorometry

Luminescent lanthanide chelates and cryptates are nowadays widely used as labels in the analysis of various biological molecules due to their enhanced detectability compared to traditional organic fluorophores using time-gated fluorescence measurement. Luminescent chelates of lanthanides (rare earths, e.g. trivalent europium, terbium, samarium and dysprosium) are an exceptional group of photoluminescent compounds [Chem. Soc. Rev. 2005; 34:1048-1077]. The lanthanide ions themselves have a very low absorption and, in addition, the excited state of the lanthanide is efficiently quenched by coordinated water molecules. Thus, the only practical solution to their excitation is to use a coordinating ligand comprising a light harvesting moiety, such as an organic antenna chromophore in the intrinsically luminescent lanthanide(III) chelate. In practice, the photoluminescence efficiency (product of absorption coefficient and quantum yield) of the lanthanide ion chelated to an efficient antenna ligand, displacing all the coordinating water molecules, can be readily enhanced up to 100 000-fold compared to a bare ion. Further, the distinct emission bands characteristic to lanthanide ion enable simultaneous measurement of up to four different lanthanides with minimal spectral crosstalk.

The excitation mechanism of lanthanide(III) chelates, where an organic light harvesting antenna is used to excite the emissive lanthanide(III) ion via energy-transfer, is exceptional among fluorescent reporters [J. Fluoresc. 2005; 15: 529-542]. Luminescent lanthanide(III) chelates comprise a reactive group, light-harvesting antenna and chelating groups, which chelate the lanthanide(III) ion through coordination bonds. The organic light harvesting chromophore is first excited from ground singlet state (S₀) to first singlet state (S₁) by light absorption, and the chromophore undergoes transition to triplet state (T₁) by intersystem crossing (ISC). The triplet state of the antenna chromophore can transfer the excitation energy to appropriate 4f energy level of the lanthanide(III) ion. Thereafter, the lanthanide ion produces characteristic f-f transition luminescence with distinct emission bands and with a long luminescence lifetime due to forbidden transition.

The chelate complexes of metals (coordination compounds) are formed through binding of ligand (or chelating molecule) to metal ion through coordinated groups. The total number of points of attachment of the ligand to the central metal ion is termed the coordination number. The ligands can be characterized for points of attachment, listing them as monodentate, bidentate, etc., where the concept of teeth (dent) reflects the number of atoms bonded to the metal centre in the chelate Cyclic structures reducing the freedom of conformations of the binding ligand often result in higher stability constants. Determination of stability constants for europium(III) complexes is described e.g. in J. Chem. Soc., Dalton Trans. 1997; 1497-1502.

Lanthanide ions complexed to a suitable ligand (e.g. aminopolycarboxylic acid) containing organic light harvesting antenna moiety or chromophore possess unusual fluorescence characteristics compared to conventional fluorophores: large Stokes shift (150-300 nm), narrow and distinct emission bands characteristic to lanthanide ion, and long luminescence lifetime (up to 2000 microseconds) [Crit. Rev. Clin. Lab. Sci 2001; 38: 441-519]. Several intrinsically fluorescent lanthanide chelates have been developed [Angew. Chem. Int. Ed. Engl. 1987; 26: 1266-1267; Bioconjugate Chem. 1994, 5, 278; Chim. Acta 1997; 80: 372-387; Anal. Chem. 2003; 75: 3193-3201; J. Chem. Soc., Perkin Trans. 2, 2000; 1281-1283; Inorg. Chem. Comm. 2002; 5: 1059-1062; JACS 1995; 117: 8132-8138; and PCT Int. Appl. WO 2005/021538]. These stable, luminescent lanthanide complexes include both cryptates and highly luminescent chelates (mainly linear or cyclic aminopolycarboxylic based chelating structures) for several lanthanides (e.g. europium(III), terbium(III), samarium(III) and dysprosium(III). The chelating ligands are designed to combine a moderately strong or strong binding of the lanthanide(III) ion and light-harvesting part to the one and same molecule. In most of the chelates, the light-harvesting (energy-absorbing) and mediating part is composed of derivatized pyridine or pyridine manifold. Some antenna structures contain other heteroatomic conjugated ring structures such as pyrazole. The light harvesting structures have typically their absorption maxima between 300 - 380 nm. In addition to the lanthanide ion, light-harvesting organic moiety and carrier ligand, the intrinsically luminescent lanthanide complexes used for labeling contain a reactive group for covalent conjugation.

The exceptional fluorescence lifetime of lanthanides enables efficient background separation by selection of such a temporal delay and gate windows (both typically tens or hundreds of microseconds) that detection is performed only when the background fluorescence (short living) has decayed away, while the lanthanide luminescence is still reasonable intense. Moreover, the large Stokes shift and the narrow emission bands enable efficient wavelength filtering to spectrally select the lanthanide luminescence, resulting in highly sensitive reporter technology (equal performance to enzyme amplified chemiluminescence) and possibility for multiparametric measurement. The technology utilizes dedicated detection method known as (microsecond) time-resolved fluorometry [Clin. Chem. 1983; 29: 65-68]. The long-lifetime fluorescence of luminescent lanthanide chelates is typically excited at ultraviolet or blue visible light [Angew. Chem. Intl. Ed. Engl. 2004; 43: 5010-5013] and the emission is detected at green and red visible wavelengths. In case of erbium, neodymium and ytterbium the excitation can be at visible wavelengths and the emission at visible or at infrared wavelengths [Chem. Phys. Lett. 1997; 276: 196-201].

### Time-Resolved Fluorescence Resonance Energy Transfer

Time-resolved fluorescence resonance energy transfer (TR-FRET) based assay methods utilizing different photoluminescent lanthanide cryptates and chelates as donors have been introduced to largely solve the major problems associated with conventional FRET-based homogeneous assays [Clin. Chem. 1993; 39:1953-1959; Anal. Biochem. 1994; 218:1-13; Proc. Natl. Acad. Sci. USA 1994; 91:10024-10028; Cytokine 1998; 10:495-499; PCT Int. Appl. WO 98/15830; US Pat. No. 5,998,146; PCT Int. Appl. WO 87/07955; Clin. Chem. 1999; 45:855-61]. These methods provide significant advantages compared to the conventional methods due to long-lifetime emission and large Stokes’ shift, but the specificity in signal generation is still limited by the radiative energy transfer (absorption of donor emission by acceptor), especially when the labeled probes are present in high concentration (e.g. to achieve a large dynamic range, or to facilitate binding in case of weak interactions) [Spectrochim. Acta, Part A, 2001; 57]. Excess of the unbound acceptor result in slowly-decaying radiative background signal at the acceptor-specific wavelength, but also the donor cross-talk at the measurement wavelength can increase the background signal unless sufficient spectral resolution is used. The utilization of non-overlapping acceptor (non-overlapping FRET) with lanthanide chelate donor [Anal. Chem. 2005; 77:1483-1487; Anal. Chim. Acta 2005; 551: 73-78] can further eliminate the possible background through reabsorption of donor emission.

In TR-FRET based assays the use of long-lifetime fluorescent lanthanide chelate (or cryptate) as a donor in combination with a conventional, short-lifetime fluorescent acceptor [Clin. Chem. 1993; 39: 1953-1959; Clin. Chem. 1999; 45: 855-861] enables specific, time-gated detection of the the energy-transfer excited sensitized acceptor emission and its discrimination from the short-lifetime, directly-excited fluorescence of the acceptor and also the background fluorescence. The crosstalk of the donor emission to the acceptor emission wavelength is also mostly quite efficiently avoided due to the narrow “line like” emission bands of the donor emission.

Homogeneous fluorescence resonance energy-transfer based nucleic acid hybridization assays utilize typically either a quenched oligonucleotide hybridization probe (donor and quencher in a cleavable oligonucleotide probe as in US Pat. No. 5,538,848 or donor and acceptor in a molecular beacon type stem-loop oligonucleotide probe that is opened upon hybridization as in US Pat. No. 5,925,517 and US Pat. No. 6,150,07) or a energy-transfer hybridization probe pair comprising separate donor and acceptor labeled hybridization probe oligonucleotides (binary probe), which hybridize next to each other to adjacent positions on complementary target sequence [Am. J. Pathology 1998; 153: 1055-1061; Bioconjugate Chem. 2002; 13: 200-205.]. The donor fluorophore is excited at its excitation wavelength and the resonance energy-transfer excited sensitized acceptor emission, which is dependent on the hybridization of the probes to adjacent positions on the target nucleic acid sequence, is detected at wavelength of acceptor emission.

Although, fluorescence resonance energy-transfer is an extremely versatile technology, the intensity of sensitized acceptor emission in hybridization probe-pair based assay can be limited by energy-transfer efficiency as efficient energy-transfer typically requires distances of just few nanometers or less. In TR-FRET based hybridization probe pair assays, however, further optimization of the distance between the donor and acceptor labels is required to avoid too efficient energy-transfer, that would hamper the time-gated detection in micro - millisecond time region due to too rapid decay of the sensitized acceptor emission. Also the hybridization probe concentration cannot be increased that much as the background through reabsorption of the donor emission will increase and limit the sensitivity and dynamic range of the assay. The quenched probe based assay, on the other hand, requires specific labeling with two different dyes and is dependent on the specificity of only a single hybridization event.

### Chelate Complementation

Lanthanide chelate complementation is a binary probe technique that is based on target-directed formation of fluorescent lanthanide complexes. In chelate complementation based hybridization assay the binary oligonucleotide probe pair comprises two oligonucleotide probes: one oligonucleotide probe is labeled with non-fluorescent lanthanide chelate and the other with light-harvesting sensitizer antenna ligand, that is able to coordinate to the lanthanide ion in the carrier chelate forming a luminescent lanthanide complex only when the both oligonucleotide probes are bound in close proximity or adjacent sequences in the nucleic acid template. First example of such template-directed self-assembly was based on DTPA-terbium(III) as carrier chelate and salicylate as light harvesting ligand [Angew. Chem. Int. Ed. Engl. 1990; 29: 1167-1169]. This approach has been used later [Anal. Biochem. 2001; 299: 169-172; J. Fluoresc. 2005; 15: 559-568] and improved to provide better sensitivity and suitability to e.g. high temperature conditions by utilizing additional lanthanide ion chelating ligand and/or cyclic lanthanide carrier chelate [Anal. Chem. 2010; 82: 751-754; Anal. Chem. 2011; 83: 9011-9016; PCT Int. Appl. WO 2010/109065]. The technique enables complete switching of the the lanthanide luminescence from dark state (non-luminescent state) to bright state (luminescent state) (or vice versa) upon presence of the target and no significant fluorescence background is observed at dark state, in contrast to prior art methods, where the modulation of lanthanide luminescence has been very limited due to observable background fluorescence.

For the luminescence binary hybridization probe assay based on lanthanide chelate complementation the probe sequences (including reporter moiety conjugation position and attachment of linker) and linkers (length and composition, including orientation and rigidity) are selected so that the two parts of the reporter, i.e. the ion carrier chelate and the antenna ligand, are brought to close proximity at correct orientation to enable self-assembly of the mixed chelate. When the complex is formed fluorescence is excited at one wavelength and the emission is measured at another wavelength using time-resolved fluorometry, i.e. using time-gated fluorescent detection after excitation pulse. Formation of the mixed chelate complex by complementation of the carrier chelate with the antenna ligand requires molecular contact at correct orientation between the antenna ligand and the central lanthanide ion. The process is actually self-assembling, as high effective local concentration favours binding even through weak coordination interactions, when both the carrier chelate and the antenna ligand are anchored into close proximity. The coordination of the antenna ligand to the ion carrier chelate provides also additional co-operative stability factor to the hybrization of the oligonucleotide probe pair and thus results in improved the specificity of the binding compared to e.g. conventional FRET-based signal generation techniques used in binary probes.

### MicroRNA Detection

MicroRNA (miRNA) are short (around 22 nucleotides) noncoding ribonucleic acids (RNA) that regulate messenger RNA (mRNA) expression and protein translation in mammals [Nat. Rev. Genet. 2004; 5: 522-531 and Nat. Rev. Immunol. 2016; 16: 279-294]. They play critical roles in many cellular processes including cell differentiation, proliferation and apoptosis. The lengths of different mature miRNA has been described to vary from 17 to 25 nucleotides and some variation in length even of miRNA produced from the same origin have been observed [Nucleic Acids Res. 2011; 39: 257-268].

miRNAs are transcribed from genome as long primary miRNA (pri-miRNA) transcripts by RNA polymerase II. miRNA maturation begins with cleavage of the pri-miRNAs by RNAse III to release approximately 70-nucleotide hairpin-shaped structures, called precursor miRNAs (pre-miRNAs) [PLoS Biol. 2005; 3: e235]. Pre-miRNAs are then actively exported to the cytoplasm. In the cytoplasm, pre-miRNAs are subsequently cleaved by another RNase III enzyme into approximately 22-nucleotide miRNA duplexes. Only one of the two strands, called mature miRNA, is predominantly transferred to the RNA-induced silencing complex (RISC), which mediates either cleavage of the target mRNA or translation silencing. The other strand is released and degraded.

miRNA are evolutionarily conserved from plants to mammals and negatively regulate gene targets by inhibiting protein translation or enhancing mRNA degradation. Body fluids, including plasma and serum, contain specific miRNA that are altered in various diseases [PLoS One 2012; 7: e41561] and there is growing interest in using these circulating miRNA as biomarkers for progression of diseases in humans [Front. Immunol. 2017; 8: 118]. This is further facilitated by the stability of miRNA in body fluids [Bioinformatics 2015; 13: 17-24]. There is thus need for methods for sensitive and specific detection and quantitation of miRNAs from different clinical samples. The spatial distribution of miRNAs is also detected and studied by in situ hybridization of cryosections of different tissues [Nat. Protoc. 2007; 2: 1508-1514]. The challenges in miRNA detection, however, are the low affinity of conventional RNA and DNA oligonucleotide probes used for their detection due to the short length of mature miRNA and the range of concentration of miRNA in blood, plasma and serum varying from nanomolar to femtomolar concentrations. To improve the oligonucleotide probe affinity (and result in higher melting temperature) towards miRNA analytes, locked nucleic acid (LNA) nucleotides have been often incorporated into DNA oligonucleotide probes [Int. J. Mol. Sci. 2015; 16: 13259-13286].

Utilization of miRNAs as biomarkers requires detection of specific miRNA targets at nanomolar and smaller concentrations [Lab. Invest. 2019; 99: 4523-469], which is often beyond the reach of direct hybridization assays based on conventional fluorescence. Most miRNA detection methods are thus enzymatic amplification based methods, which rely e.g. padlock probe hybridization and ligation prior to rolling circle amplification (RCA) (miRNA acting as primer) [RNA 2006; 12: 1747-1752], direct target primed rolling circle amplification of circular probe [Anal Chem 2014; 86: 1808-1815] and target based toehold-initiated rolling circle amplification of circular seal probe [Angew. Chem. Int. Ed. Engl. 2014; 53: 1389-2393]. Padlock probe hybridization, nick ligation and target-primed RCA amplification has been also combined with TR-FRET-based binary probe detection of the amplified product [Chem. Sci. 2018; 9: 8046-8055] to reach sub-picomolar detection limits. Ligation of two stem-loop probes bound to miRNA target and subsequent PCR amplification of the ligated probes have also been demonstrated [Talanta 2011; 85: 1760-1765]. Widely used real-time quantitative PCR based methods employ target-specific stem-loop reverse transcription primers that bind to 3′ portion of miRNA to generate DNA templates, that are further amplified by PCR with additional miRNA specific forward primer and monitored with Taqman probes [Nucleic Acids Res. 2005; 33: e179; Genes (Basel) 2016; 7: 131]. These methods, however, are challenged by the short length of the miRNA sequence in the product, which makes it difficult to fit the miRNA specific dye-labeled Taqman probe completely in the miRNA sequence region still between the primer regions. Further, the short recognition sequence of the stem-loop reverse transcriptase primer can result in reverse transcription of also closely related miRNAs and thus reduced specificity or inefficient reverse transcription of the preferred miRNA [Chem. Sci. 2018; 8046-8055]. Direct detection of miRNA has been demonstrated also e.g. using binary probe approach and quantum dots and Tb-cryptate as FRET-pair achieving nanomolar detection limit [ACS Nano 2015; 9: 8449-8457], but improved sensitivity in direct miRNA detection would still be preferred. The low miRNA concentration in the body fluids, however, can be partly circumvented by concentrating miRNA by e.g. immunoprecipitation to capture circulating miRNAs that are protein bound, collecting RISC complexes, and extracellular microvesicles (EVs) or exosomes that contain and transport miRNA in biological fluids [Oncotarget 2016; 7: 75353-75365].

The single stem-loop reverse transcriptase (RT) primers widely utilized in assay for mature miRNAs provide improved selectivity and affinity compared to linear primers, but in presence of large excess of related pri-miRNAs or pre-miRNAs some low level of reverse transcription of them can still happen [Curr. Protoc. Mol. Biol. 2011; unit 15.10]. The steric hindrance provided by the single-stem RT primer at one end of the miRNA of is thus beneficial, but not yet complete solution. It is known that the positioning the stem-loop primer to the target is important for the selectivity. The best amplification efficiency is obtained, when there is nick (i.e. no gap or overlap) formed between the 3′ terminus of the target and the 5′-terminus of stem-loop primer, but even in presence of overlapping binding sites or gap between the terminuses (i.e. absence of nick) some extension can still occur [RNA 2013; 19: 1-10]. The method has also other limitations, since it detects also double-stranded RNA templates even in absence of denaturation due to strand-displacement activity of the reverse transcriptase.

There are also multiple single stem-loop probe based approaches, where the probe is actually a template for initial extension of the miRNA by RNA extension enzyme [Anal. Chem. 2018: 7107-7111; Sci. Reports 2017; 7: 11396]. These methods, however, are not better in selectivity to the mature miRNA compared to the single stem-loop RT primer based methods.

Binary stem-loop probe based ligation assays have been earlier described for detection of mature miRNA targets [Talanta 2011; 85: 17560-1765; Anal. Chem. 2009; 81: 5446-5451]. The assays, however, utilize only stem-loop probes that don’t produce nicks between the probes and the target. In the first assay, this is actually avoided intentionally by design by adding noncomplementary bases to the end of the stem sequence of the probe. In the second assay, the stem structure in the probes is actually opened upon hybridization to the target and no stem structure is anymore present, when the oligonucleotide probes are hybridized to the target or ligated together. The complementary region of the oligonucleotide probe towards the fragment of target nucleic acid is thus part of the stem structure. Further, immobilized single stem-loop oligonucleotide probe forming a nick between the probe and labeled target has been utilized earlier in heterogeneous hybridization microarray combined with ligation step to join the nick structure [Nucleic Acids Res. 2001; 29: e92]. There is however no second oligonucleotide probe employed and thus the assay does not utilize binary probe.

Unfortunately, the current direct and amplification based nucleic acid hybridization assay methods have limited specificity and/or sensitivity to detection of individual microRNAs, and not all methods are able to discriminate the mature microRNAs (miR) from pre-miRNA, pri-mRNA and genomic sequences coding the microRNAs.

Accordingly, the purpose of the present invention is to provide a highly sensitive and specific hybridization assay method without amplification step to directly detect and/or quantitate of a short nucleic acid analyte molecule with defined sequence and length, such as mature miRNAs, avoiding the discussed disadvantages and problems observed with the current methods intended for direct detection of microRNAs.

### OBJECT AND SUMMARY OF THE INVENTION

One object of the present invention is to provide a bioassay method for detecting and/or quantitating a short single-stranded nucleic acid analyte molecule.

In a first aspect, the present invention provides a bioassay method for detecting and/or quantitating a nucleic acid analyte employing a binary probe system, the method comprising a step of contacting two oligonucleotide probes with a sample, wherein
(a) a first oligonucleotide probe comprises a double-stranded terminal stem-loop structure and a single-stranded terminal sequence overhang that is complementary to and is capable of selectively hybridizing with a first region of the nucleic acid analyte, and
b) a second oligonucleotide probe comprises a single-stranded terminal sequence that is complementary to and is capable of selectively hybridizing with a second region of the nucleic acid analyte, and
said first region of the nucleic acid analyte is terminal region and said single-stranded terminal sequence overhang of said first oligonucleotide probe hybridizes to said first region of the nucleic acid analyte, and said hybridization results in formation of a first nick structure between a terminus of said nucleic acid analyte and a first terminus of said first oligonucleotide probe, and said first terminus of said first oligonucleotide probe is part of said double-stranded terminal stem-loop structure of said first oligonucleotide probe, wherein said first and second region of the nucleic acid analyte are strictly adjacent regions of said nucleic acid analyte, and said single-stranded terminal sequences of said first and second oligonucleotide probe hybridize to said nucleic acid analyte forming a second nick structure between a second terminus of said first oligonucleotide probe and a first terminus of said second oligonucleotide probe;

and detecting the presence or absence of said nucleic acid analyte bound to said first and second oligonucleotide probes, wherein the presence of said nucleic acid analyte bound to said first and second oligonucleotide probes confirms the presence of the analyte in the sample.

According to a second aspect, the present invention provides a bioassay method for detecting and/or quantitating a nucleic acid analyte employing a binary probe system, where at least one of the two discrete oligonucleotide probe parts of the binary oligonucleotide probe system has partially double-stranded (self complementary) stem-loop structure at one terminus and single-stranded overhang region at the other terminus, where the single-stranded regions of both discrete parts of the binary probe system hybridize to regions i) in close proximity or ii) strictly adjacent in the sequence of the single stranded nucleic acid analyte molecule and at least one discrete part of the binary oligonucleotide probe system comprising a stem-loop structure hybridizes to a terminal region in the sequence of the single-stranded nucleic acid analyte molecule forming a nick between the end of the stem-loop structure of the oligonucleotide probe and a terminus of the nucleic acid analyte molecule.

According to a particular embodiment, both the two discrete oligonucleotide probe parts of the binary oligonucleotide probe system have partially double-stranded (self complementary) stem-loop structure at one terminus and single-stranded overhang region at the other terminus and the single-stranded regions of the both discrete parts of the binary oligonucleotide probe system hybridize to terminal regions strictly adjacent in the sequence of the single-stranded nucleic acid analyte molecule forming either two or three nick structures, where in the latter case two of the nicks are at opposite terminuses of the nucleic acid analyte molecule, between the terminuses of the nucleic acid analyte molecule and the ends of the stem-loop structures of the oligonucleotide probes, and the third nick, when the distinct oligonucleotide probes hybridize to strictly adjacent positions in the nucleic acid analyte, is formed between the terminal overhangs of the oligonucleotide probe.

According to another particular embodiment, the binary probe system employed in the bioassay method is based on a luminescent reporter technology, either lanthanide chelate complementation or resonance energy transfer with lanthanide label as a donor. Thereby the method allows detection and/or quantitation of the short nucleic acid analyte molecule by time-gated fluorescence measurement.

According to yet a further aspect, the invention provides a kit for detecting and/or quantitating a short nucleic acid analyte molecule. The kit includes the two discrete oligonucleotide probe parts of the binary oligonucleotide probe system:
(a) a first oligonucleotide probe comprising a double-stranded terminal stem-loop structure and a single-stranded terminal sequence overhang that is complementary to and is capable of selectively hybridizing with a first region of a single-stranded nucleic acid analyte, and
b) a second oligonucleotide probe comprising a double-stranded terminal stem-loop structure and a single-stranded terminal sequence overhang that is complementary to and is capable of selectively hybridizing with a second region of said single-stranded nucleic acid analyte,
wherein said first and second region of the single-stranded nucleic acid analyte are strictly adjacent regions of said single-stranded nucleic acid analyte,

wherein a terminal nucleotide in a terminus of the single-stranded terminal sequence overhang of said first oligonucleotide probe and a terminal nucleotide in a terminus of the single-stranded terminal sequence overhang of said second oligonucleotide probe are designed to bind to adjacent nucleotides in said single-stranded nucleic acid analyte in order to form a first nick structure between said terminal nucleotides of the single-stranded terminal sequence overhangs of said first and second oligonucleotide probes when said probes are bound to said single-stranded nucleic acid analyte, said first nick structure lacking a phosphodiester bond between a pair of adjacent nucleotides bound to a complementary strand; and

wherein said single-stranded terminal sequence overhangs of said first and second oligonucleotide probes are further designed so that when said probes are bound to said single-stranded nucleic acid analyte, one of the terminal nucleotides of said single-stranded nucleic acid analyte forms a second nick structure with a terminal nucleotide in a terminus which is part of said double-stranded terminal stem-loop structure of the first oligonucleotide probe and another of the terminal nucleotides of said single-stranded nucleic acid analyte forms a third nick structure with a terminal nucleotide in a terminus which is part of said double-stranded terminal stem-loop structure of the second oligonucleotide probe, said second and third nick structures lacking a phosphodiester bond between a pair of adjacent nucleotides bound to a complementary strand..

According to a yet another particular embodiment, the short nucleic acid analyte molecule is mature microrna with defined length and composition of the sequence.

### BRIEF DESCRIPTION OF THE DRAWINGS

FIG. 1 illustrates a binary probe hybridization assay based on partially double-stranded stem-loop probe together with conventional probe, and hybridization of the two probes in close proximity to each other to the target nucleic acid. The hybridization of the stem-loop probe is dependent on the defined termination sequence of the target nucleic acid at one terminus.

FIG. 2 illustrates a binary probe hybridization assay based on partially double-stranded stem-loop probe together with conventional probe, and hybridization of the two probes strictly adjacently to the target nucleic acid. The hybridization of the stem-loop probe is dependent on the defined termination sequence of the target nucleic acid at one terminus, and hybridization of the conventional probe is dependent on the formation of nick between the two probes hybridized on the target nucleic acid.

FIG. 3 illustrates a binary probe hybridization assay based on partially double-stranded stem-loop probe pair, and hybridization of the probe pair strictly adjacently to the target nucleic acid. The hybridization of the probe pair is dependent on the defined termination sequence of the target nucleic acid at both terminuses and the defined length of the target nucleic acid.

FIG. 4 illustrates an example of a luminescence binary probe hybridization assay based on chelate-complementation and partially double-stranded stem-loop probe pair, and formation of a fluorescent complex upon hybridization of the probe pair adjacently to the target nucleic acid. The hybridization of the probe pair is dependent on the defined termination sequence of the target nucleic acid at both terminuses and the defined length of the target nucleic acid.

FIG. 5 illustrates an example of a luminescence binary probe hybridization assay based on chelate-complementation and partially double-stranded stem-loop probe together with conventional probe, and formation of a fluorescent complex upon hybridization of the two probes next to each other to the target nucleic acid. The hybridization of the stem-loop probe is dependent on the defined termination sequence of the target nucleic acid at one terminus and hybridization of the conventional probe is dependent on the formation of nick between the two probes hybridized on the target nucleic acid.

FIG. 6 illustrates an example of a luminescence binary probe hybridization assay based on chelate-complementation and partially double-stranded stem-loop probe pair, and formation of a fluorescent complex upon hybridization of the probe pair in close proximity to the target nucleic acid. The hybridization of the probe pair is dependent on the defined termination sequence of the target nucleic acid at both terminuses.

FIG. 7 illustrates an example of a luminescence binary hybridization assay based on fluorescence resonance energy transfer and partially double-stranded stem-loop probe pair, and formation of a fluorescent resonance energy-transfer complex upon hybridization of the probe pair adjacently to the target nucleic acid. The hybridization of the probe pair is dependent on the defined termination sequence of the target nucleic acid at both terminuses and the defined length of the target nucleic acid.

FIG. 8 illustrates an example of a solid-phase luminescence binary probe hybridization assay based on chelate-complementation and partially double-stranded stem-loop probe pair, where one of the probes is immobilized on the solid support, and formation of a fluorescent complex upon hybridization of the probe pair in close proximity to the target nucleic acid. The hybridization of the probe pair is dependent on the defined termination sequence of the target nucleic acid at both terminuses. The fluorescent complex is formed in the surface area of the solid-support, where one of the probes is immobilized.

### DETAILED DESCRIPTION OF THE INVENTION

Current bioassay methods to detect and/or quantify short nucleic acid targets with defined base sequence and length such as mature microRNAs are limited in specificity and sensitivity. The inventor has found that a nick forming binary probe system based on at least one stem-loop hybridization probe with complementary overhang to the nucleic acid target combined with time-resolved fluorescence resonance energy transfer label pair or formation of lanthanide chelate complex provides a unique approach to sensitive and specific measurement of short sequence nucleic acid analytes such as mature microRNAs with defined nucleobase sequence and length about 22 nt without use of enzymatic nucleic acid amplification or extension.

In addition to the sensitive measurement of short sequence nucleic acid analytes that is challenging with conventional fluorescence based methods, the invention provides improved specificity to the nucleic acid analytes of defined length, nucleobase sequence composition and also terminal nucleobase sequences. The invention solves how to directly detect and quantify defined short mature microRNAs without interference of other closely related nucleic acids such as pre-miRNA, pri-miRNA and genomic DNA, that can even comprise the same base sequence as part of their longer sequence. The invention further provides high hybridization selectivity to sequence of the target nucleic originating from the base stacking effect, which increases the melting temperature due to formation of one or multiple nick structures in perfectly matching probe hybridization. The formation of the mixed chelate complex is further introduced to increase the stabilization effect based on the co-operative binding of binary probe system.

Binary stem-loop probe system provides surprising advantages in the detection of short nucleic acid target. First, due to base-stacking effect and nick formation between the target and the probe, it improves the probe affinity and enables sensitive detection of short nucleic acid target that is not possible with linear probes. Second, it provides strict selectivity to the length of product and terminal nucleobase sequence that is not possible with linear probes. Third, the method enables unbiased, direct and amplification free detection of the target in homogeneous assay format, and fourth, the method can also be used for multiplexed array based spatially resolved detection and additional mode of multiplexing is based on the fluorescence color. The stem-loop binary probe concept has not been described even both stem-loop probes (also called hairpin probes) [Nucleic Acids Res 2001; 29: 996-1004] and binary probes as such have been known for decades [Trends in Biotech 2002; 20: 249-256]. In most nucleic acid assays the targets are significantly longer than in the case of mature miRNA and therefore in other applications there is no need to such strict discrimination based on both the target length and sequence composition that is provide by binary stem-loop probe system. The combination of two stem-loop oligonucleotide probes in a stem-loop binary probe makes the system also superior to conventional single-stranded linear probe in target hybridization. The contiguous base stacking interaction [Nucleic Acids Res 2006; 34: 564-574] around the formed nick structures, i.e. between double-stranded stem of the probe and duplex formed with perfectly matched single-stranded target, provide additional free energy minimization, increasing the stability of the resulting probe-target complex. This extra stabilization is lacking in case of mismatched target and, thus, hybridization specificity is improved, i.e. the method provides better discrimination between matched and mismatched target. Further, the hybridization kinetics of the stem loop probes is also faster than with linear probes [Nucleic Acids Res 2001; 29: 996-1004] providing yet another beneficial feature to the present invention. It is surprising, that the stacking interactions of nucleobases have such significant large role in the structural stability of nucleic acids in aqueous solution [Nucleic Acids Res. 1993; 21: 2051-2056] compared to the hydrogen bonds between the bases.

Conventional fluorescence based assays are not able to provide adequate sensitivity to direct detection of microRNAs and thus the state of the art assays are based on enzymatic nucleic acid amplification steps either utilizing target as template or primer [Lab. Invest. 2019; 99: 452-469]. Time resolved fluorescence based FRET-assays have been demonstrated, but these methods are also amplification based [ACS Nano 2015; 9: 8449-8457] or utilize conventional linear probes and there is no concurrent binding of two oligonucleotide probes to the target as in case of binary probes [Chem. Sci. 2018; 9: 8046-8055].

In scientific literature a binary stem-loop probe based ligation assay has been earlier described for detection of mature miRNA targets [Talanta 2011; 85: 17560-1765], but it has a major difference to the present invention. The assay is based on formation a nick structure only between the overhangs of the stem-loop probes hybridized on the complementary RNA template and ligation of the nick preferably with T4 RNA ligase 2. The assay utilizes only stem-loop probes that don’t produce nicks between oligonucleotide probe and the target and, thus, don’t benefit of the similar base stacking effect as in the present invention. The nick formation in the assay is actually avoided intentionally by design by adding noncomplementary bases to the end of the stem sequence most likely to avoid the formation of the nick and avoid possible ligation. This is completely in contrast to the present invention and teaches not to utilize the advantage of stem-loop probes in the binary probe hybridization assay. Further, the ligated binary probe is not directly detected based on fluorescence, but the probes act as template for further quantitative PCR reaction that is monitored with double-stranded DNA intercalating dye.

Molecular beacon type stem-loop oligonucleotide probes [Angew. Chem. Int. Ed. Engl. 2009; 48: 856-870] are widely used luminescence hybridization bioassays, but these are not binary probes and in contrary to the present invention they contain the complementary sequence towards the target nucleic acid in the loop region between the complementary stem regions of the sequence. In the present invention the complementary sequence towards the target nucleic acid fragment is a terminal overhang sequence in the stem-loop oligonucleotide probe and there is no single stranded gap sequence between the one of the complementary stem regions of the probe oligonucleotide sequence and the complementary sequence towards the target, which enables formation of the nick structure upon. Further, in the present invention the stem structures of the probes are not opened upon hybridization.

The stem-loop based binary probes are able to bind shorter single stranded nucleic acid targets than possible with linear binary probe system. Shorter complementary sequences resulting in higher melting temperature can be used due to stabilization effect of base stacking within double stranded region, when the terminal sequence of the target hybridizes next to double-stranded stem region of the probe (resulting nick structure). The especially suitable targets are short single stranded nucleid acid analytes such microRNAs (miRNA, single stranded, length between 18-24 nt, most commonly 22 nt). When the binary probe comprises a lanthanide chelate complementation label pair, the adjacent binding of the two parts of the stem-loop based binary probe system to the target nucleic acid fragment results in self-assembly of mixed lanthanide chelate complex by coordination binding of the antenna chromophore to the lanthanide ion in the carrier chelate, and renders the binary probe system luminescent. The adjacent hybridization events are either in immediate vicinity, strictly adjacent i.e. 0 nt between the adjacent recognized sequences forming a nick structure, or there is 1 - 20 nt non-recognized sequence gap on target between the proximal recognized sequences. Simultaneous binding of the both parts of the binary probe system to immediately adjacent positions (nick structure formed between the probe overhangs hybridized to the target) and the self-assembly of the mixed lanthanide chelate complex (coordination bonds) strengthen together the melting temperature and improve the specificity of the target detection due to both base stacking effect and antenna ligand coordination to ion carrier chelate.

Switchable lanthanide luminescence is a sensitive luminescence reporter technology, which has been found superior method for homogeneous nucleic acid hybridization assays. In the present invention it is further combined with the stem-loop binary oligonucleotide probe system to improve the detectability of short single stranded nucleic acid targets such as mature microRNAs. The detection with binary probe system is more specific than with any single probe based systems since simultaneous binding of two discrete parts of the probe system are required for signal generation and the specificity is further enhanced by the formation of nick structures and enhanced affinity provided by base stacking.

Embodiments of the invention enable thus specific and sensitive detection of mature microRNAs without amplification even from clinical samples with high autofluorescence background due to time-gated fluorescence detection of switchable lanthanide luminescence.

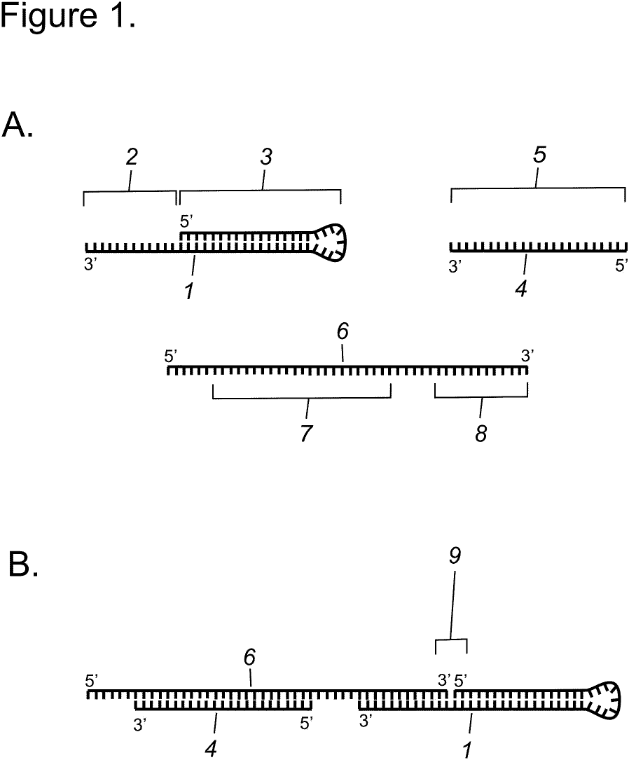

The alternative embodiments of the invention are described in FIGS. 1 - 3.

FIG. 1 (A) describes a hybridization assay based on a binary oligonucleotide probe system, comprising two oligonucleotide probes, wherein a first oligonucleotide probe (1) comprises a double-stranded 5′ terminal stem-loop sequence (3) and a single-stranded 3′ terminal overhang sequence (2) that is complementary to and is capable of selectively hybridizing with a 3′ terminal region (8) of the nucleic acid analyte (6), and a second oligonucleotide probe (4) comprises a single-stranded 5′ terminal sequence (5) that is complementary to and is capable of selectively hybridizing with a another region (7) of the nucleic acid analyte (6) non-overlapping with the 3′ terminal region (8), where the single stranded 3′ terminal sequence of the first oligonucleotide probe hybridizes. (B) Both first and second oligonucleotide probe hybridize with their complementary regions of the nucleic acid analyte (6). The hybridization of the first oligonucleotide probe (1) results in formation of a stabilizing nick structure (9) comprising 3′ terminus of the nucleic acid analyte (6) and 5′ terminus of the first oligonucleotide probe (1).

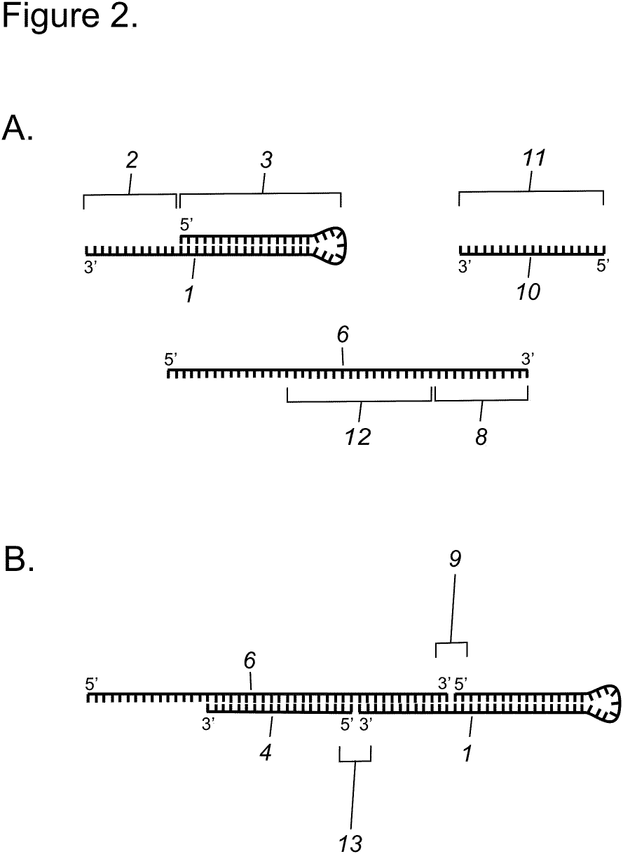

FIG. 2 (A) describes a hybridization assay based on a binary oligonucleotide probe system, comprising two oligonucleotide probes, wherein a first oligonucleotide probe (1) comprises a double-stranded 5′ terminal stem-loop sequence (3) and a single-stranded 3′ terminal overhang sequence (2) that is complementary to and is capable of selectively hybridizing with a 3′ terminal region (8) of the nucleic acid analyte (6), and a second oligonucleotide probe (10) comprises a single-stranded 5′ terminal sequence (11) that is complementary to and is capable of selectively hybridizing with a another region (12) of the nucleic acid analyte (6) in adjacent position to the 3′ terminal region (8), where the single stranded 3′ terminal sequence of the first oligonucleotide probe hybridizes. (B) Both first and second oligonucleotide probe hybridize with their complementary regions of the nucleic acid analyte (6). The hybridization of the first oligonucleotide probe (1) results in formation of a stabilizing nick structure (9) comprising 3′ terminus of the nucleic acid analyte and 5′ terminus of the first oligonucleotide probe, and due to adjacent position of the complementary regions of the two probes at the nucleic acid analyte (6), an another stabilizing nick structure (13) is formed comprising 5′ terminus of the second oligonucleotide probe (4) and 3′ terminus of the first oligonucleotide probe (1).

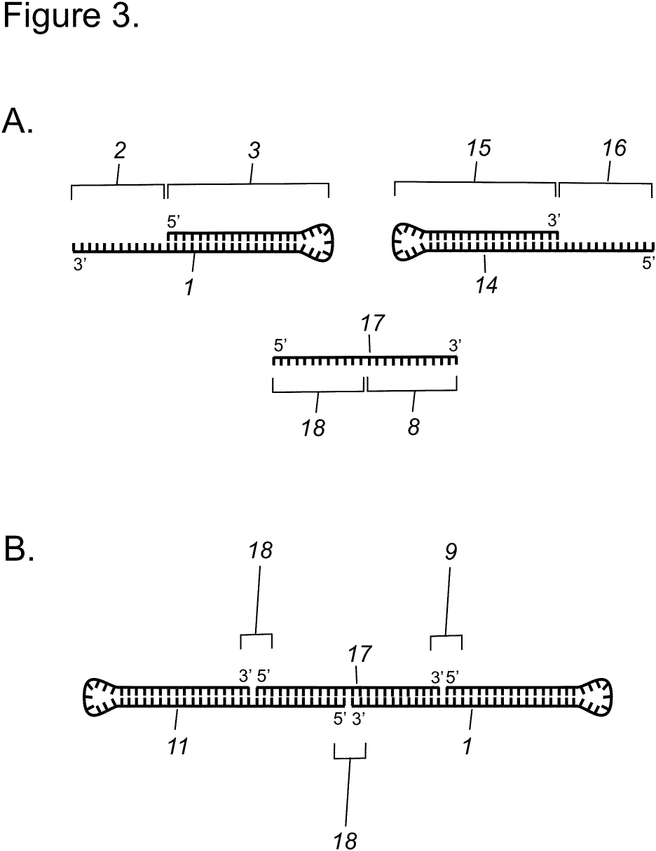

FIG. 3 (A) describes a hybridization assay based on a binary oligonucleotide probe system, wherein a first oligonucleotide probe (1) comprises a double-stranded 5′ terminal stem-loop sequence (3) and a single-stranded 3′ terminal overhang sequence (2) that is complementary to and is capable of selectively hybridizing with a 3′ terminal region (8) of the nucleic acid analyte (17), and second oligonucleotide probe (14) comprises a double-stranded 5′ terminal stem-loop sequence (15) and a single-stranded 3′ terminal overhang sequence (16) that is complementary to and is capable of selectively hybridizing with a 5′ terminal region (18) of the nucleic acid analyte (17) in adjacent position to the 3′ terminal region (8), where the single stranded 3′ terminal sequence of the first oligonucleotide probe (1) hybridizes. (B) Both first and second oligonucleotide hybridize with their complementary regions of the nucleic acid analyte (17). The hybridization of the first oligonucleotide probe (1) results in formation of a stabilizing nick structure (9) comprising 3′ terminus of the nucleic acid analyte and 5′ terminus of the first oligonucleotide probe, the hybridization of the second oligonucleotide probe (11) results in formation of a stabilizing nick structure (18) comprising 5′ terminus of the nucleic acid analyte and 3′ terminus of the second oligonucleotide probe, and due to adjacent position of the complementary regions of the two probes at the nucleic acid analyte (17), a yet another stabilizing nick structure (13) is formed comprising 5′ terminus of the second oligonucleotide probe (11) and 3′ terminus of the first oligonucleotide probe (1).

FIGS. 1 - 3 of the hybridization bioassays illustrate (A) the assay components including the binary oligonucleotide probe system and the short target nucleic acid analyte, and (B) formation of the hybridization complex, comprising one or multiple nick structures, of the binary oligonucleotide probe system upon presence of the short target nucleic acid analyte. The formation of the hybridization complex is dependent on the defined sequence composition and length of the short target nucleic acid analyte. In absence of the short target nucleic acid analyte the two discrete oligonucleotide probes of the binary oligonucleotide probe system are not hybridized, i.e. no hybridization complex and no nick structures are formed.

If the sequence composition of the short target nucleic acid is not fully complementary at the binding regions of the two discrete oligonucleotide probes of the binary probe system or the length of the short target nucleic acid does not allow formation of any of the nick structures, at least one the two discrete oligonucleotide probes of the binary oligonucleotide probe system is not hybridized. When the two discrete oligonucleotide probes are not both hybridized simultaneous on the target nucleic acid, no signal is generated by the binary probe system.

### Definitions

The terms “stem-loop” and “stem-loop structure” shall be understood here to mean a lollipop-shaped nucleic acid structure, that is also known as hairpin structure, formed when a single-stranded nucleic acid molecule comprising an intramolecular palindromic sequence, i.e. the nucleic acid molecule containing the matching sequences in 5′ to 3′ and 3′ to 5′ directions (reverse complement), loops partially back on itself to form a complementary double-stranded region (stem) topped by a single-stranded loop region comprising the nucleotides located between the matching sequences in the nucleic acid molecule.

The term “overhang” associated with the stem-loop structure shall be understood here to mean single-stranded 5′ or 3′ terminal stretch of nucleic sequence outside the stem-loop structure.

The term “stem-loop probe” shall be understood here to mean oligonucleotide probe that contains one stem-loop structure and one either 5′ or 3′ terminal overhang sequence that is entirely complementary to the fragment of target nucleic acid. The terminus that is not part of the overhang sequence is part of the double-stranded stem structure. The stem-loop probe is also known as hairpin probe.

The terms “nick” and “nick structure” shall be understood here to mean a discontinuity in double-stranded nucleic acid, where there is a gap in place of the phosphodiester bond between strictly adjacent nucleotides in one of the two nucleic acid strands of the double helix, but the other strand is intact, and the hybridized strands don’t dissociate even in presence of the nick. In the nick structure there is thus a break in one strand of double-stranded nucleic acid caused by a missing phosphodiester bond between two neighbouring (i.e. immediately adjacent in the nucleic acid sequence) nucleotides, that are both complementary in base pairing to the opposite strand. The nick structure comprises 5′ and 3′ terminus in one strand of double-stranded nucleic acid DNA and, in the opposite strand between the nucleotides complementary in base pairing to the terminal nucleotides of the mentioned 5′ and 3′ terminuses, there is no single stranded region, i.e. not even a single nucleotide without complementary nucleotide in base pairing with the other strand. The nick structure can comprise or not comprise the 5′ terminal phosphate (i.e. phosphate group coupled at 5′ terminal hydroxyl group) at the location of the missing phosphodiester bond. The 3′ terminus at the nick structure comprises typically a 3′ hydroxyl group.

The term “oligonucleotide probe” refers to labeled polynucleotide that comprises a complementary sequence to the nucleic acid analyte of interest and is used to detect and quantitate the nucleic acid analyte as part of the probe system in the oligonucleotide hybridization assay.

The term “complementary” and “complementary sequence” shall be understood here to mean nucleic acid sequence of bases that can hybridize and form double-stranded structure with other nucleic acid sequence by matching base pairs (A-T, A-U and C-G). In the double-stranded structure the sequences go in opposite directions, i.e. complementary sequence is reverse complement of the other sequence. Upon hybridization the complementary nucleic acid sequences form a double-stranded nucleic acid structure, typically a DNA double helix or an RNA-DNA duplex. Two single-stranded nucleotide sequences are typically said to be complementary, when the nucleotides of one strand, optimally aligned and compared, pair selectively with at least about 80% of the nucleotides of the other nucleic acid strand, usually at least about 90% to 95%, and more preferably from about 98 to 100%. As known to those skilled in the art, a very high degree of complementarity, preferably 100%, is needed for specificity and sensitivity involving hybridization.

The terms “binary probe”, “binary probe system” and “binary oligonucleotide probe” shall be understood here to mean pair of oligonucleotide probe molecules, i.e. two discrete oligonucleotide probe parts, that produce a detectable signal only when the both parts are hybridized to proximal or adjacent positions in the target nucleic acid molecule. The both parts of the binary probe comprise a oligonucleotide sequence that is complementary to the adjoining positions of the target nucleic acid and, in addition, functional moiety, part of label pair or label fragment that produce a signal when two parts of the binary probe hybridize on the target nucleic acid. Examples of such binary probes are split and two-component probes [Chem. Rev. (2010) 110: 4709-4723].

The terms “specifically hybridize” and “specific hybridization” and “selectively hybridizing” are used herein to mean the binding, duplexing, or hybridizing of a nucleic acid sequence preferentially to a particular complementary nucleotide sequence under stringent conditions.

The terms “labeling” and “oligonucleotide labeling” shall be understood here to mean attachment of a label moiety covalently to the oligonucleotide probe at one or multiple defined positions. The labeling of the oligonucleotide probe can be done during oligonucleotide synthesis by using an appropriate building block to introduce the label moiety only or modified nucleotide containing the label moiety to the specific position of the oligonucleotide. [Chapter “Solid-phase oligonucleotide labeling with DOTA” by Jaakkola, et al. (2007) in Current protocols in nucleic acid chemistry, edited by Beaucage, S.L. et al.; Chapter 14: Unit 4.31; online publication by John Wiley & Sons]. Alternatively labeling can be done post synthetically by coupling e.g. amino reactive label moiety to the modified nucleotide, comprising a primary amino group coupled to the nucleotide with a linker, in the synthesized oligonucleotide. In both approaches the label is coupled to oligonucleotide via linker. Combination of both during synthesis and post synthetic labeling enables introduction of different label moieties to distinct position in the oligonucleotide. Modified nucleotide building blocks, where linker is coupled to the nucleobase in a position that base can still properly hybridize to the complementary base, enable attachment of the label practically to any position of the oligonucleotide and double helix without interfering the base stacking and stability of double helix. The linker can be e.g. aliphatic carbon chain or polyethyelene oxide chain. The label moiety can be covalently attached to the using e.g. iodoacetamide, N-hydroxysulfosuccinimide, maleimide or isothiocyanate activation “click-chemistry” approaches [J. Am. Chem. Soc. (2005) 127:14150-14151; Trends Biochem. Sci. (2005) 30:26-34].

The term “solid-support” refers to an insoluble material where one of the probes can be adsorbed or immobilized utilizing similar coupling chemistries as used for labeling of the oligonucleotide probe. Known materials of this type include hydrocarbon polymers such as polystyrene and polypropylene. In addition, the solid support be composed of silica gel, silicone wafers, glass, metals. The solid support may be physically in the form of particulates, beads, tubes, slides, strips, disks or microtitration wells and plates.

The terms “fluorescence” and “luminescence” shall be understood here to cover photoluminescence, i.e. luminescence excited by light, fluorescence, including delayed fluorescence with microsecond or millisecond fluorescence lifetime, and phosphorescence. In addition, the term shall cover electrogenerated luminescence and electrochemiluminescence. The luminescence can be measured or imaged either as steady-state luminescence or as time-gated luminescence.

The terms “lanthanide” and “lanthanide ion” and “luminescent lanthanide ion” and “Ln³⁺” shall be understood here to be equivalent to “rare earth metal ion” and to include single trivalent lanthanide ions and any combination of different trivalent lanthanide ion or rare earth ion from the following: cerium, neodymium, praseodymium, samarium, europium, promethium, gadolinium, lutetium, terbium, dysprosium, holmium, erbium, thulium, ytterbium, and yttrium, especially europium, terbium, samarium, dysprosium, erbium, neodymium and ytterbium.

In this disclosure the terms “luminescent lanthanide chelate”, “fluorescent lanthanide chelate”, “luminescent lanthanide complex”, and “complemented lanthanide chelate” shall be understood to include luminescent complexes comprising a lanthanide ion carrier chelate and light-harvesting antenna ligand, where the luminescence of the lanthanide ion in the ion carrier chelate is excited through a light-harvesting chromophore or other excitable structure in the light-harvesting antenna. The luminescent lanthanide chelate can comprise the light harvesting antenna covalently joined to the ion carrier ligand. The complemented lanthanide chelate can be a mixed chelate comprising the ion carrier chelate and the light-harvesting antenna ligand bound to the lanthanide ion by coordination bonds. Examples of intrinsically fluorescent lanthanide chelates are europium(III) and terbium(III) chelates and cryptates that can be excited at wavelength range 320 - 365 nm through the chromophore that is part of the ligand and and emit at 620 nm or 545 nm depending on the lanthanide ion, respectively.

The terms “lanthanide ion carrier chelate”, “ion carrier chelate” and “carrier chelate” shall be understood to include as such essentially non-luminescent lanthanide chelate complexes and their derivatives, which comprise a chelating ligand, i.e. ion carrier ligand, and a luminescent lanthanide ion, but which do not comprise an efficient light-harvesting antenna chromophore that is essential to efficiently excite the luminescence of the lanthanide ion in the carrier chelate. Examples of lanthanide ion carrier chelates are cyclic or non-cyclic aminopolycarboxylic acid chelates of europium(III) or other luminescent lanthanide ions, where coordination number of the lanthanide ion is preferably equal to or more than 6 dentates, optimally 7 or 8, but which do not contain efficient light-harvesting antenna structure to sensitize and excite the luminescence of the lanthanide ion [PCT Int. Appl. WO 2010/109065]. Additional structures of ion carrier chelates for labelling of an oligonucleotide are illustrated e.g. in US Pat. No. 6,949,639.

The terms “complementing ligand”, “light harvesting antenna” or “antenna ligand” shall be understood to include as such essentially non-luminescent chelating ligands and their derivatives, which comprise a light-harvesting chromophore or other excitable structure and which are capable of complementing a lanthanide ion carrier chelate to form a luminescent lanthanide complex, where the luminescence of the lanthanide ion in the carrier chelate is excited through non-radiative energy transfer from a light-harvesting chromophore or other excitable structure in the antenna ligand upon either its photoexcitation or electroexcitation. Typically the antenna ligand is a monodentate, bidentate, tridentate or tetradentate ligand, most preferably bidentate or tridentate ligand, the organic light harvesting structure contains aromatic rings or heterocycles, and the light-harvesting structures has a triplet state energy level appropriate for the trivalent lanthanide ion present in the ion carrier chelate. Examples of suitable triplet state energies and light-harvesting structures for lanthanide ions are presented in PCT Int. Appl. WO 2010/109065 and J. Luminescence (1997) 75: 149-169.

The terms “FRET-pair” and “fluorescent resonance energy transfer donor and acceptor pair” shall be understood to refer to combination of a fluorescent dye (donor) and another fluorescent or non-fluorescent dye (acceptor), where i) the dyes either have spectral overlap between the emission of the donor and absorption of the acceptor to enable Förster type resonance energy-transfer from donor to acceptor, when the dyes are in close proximity, or ii) the acceptor dye is so called universal quencher that does not need spectral overlap to enable energy-transfer from donor to acceptor, when the dyes are in close proximity.

The terms “non-luminescent” and “non-fluorescent” shall be understood as a property of a light absorbing compound not to produce any or a significant amount of a desired type of luminescence, e.g. long lifetime luminescence, when excited and relaxing from the excited state. In contrast to luminescent compounds, the excited-state energy of a non-luminescent compound is predominantly relaxed via non-radiative pathways, typically producing heat instead of light, or rapid emission instead of slowly decaying emission or the excitation efficiency is weak. The molar extinction coefficient or molar absorptivity of a non-luminescent compound is very low, typically below 10 L mol⁻¹ cm⁻¹, or the fluorescence quantum yield of a non-luminescent compound is very poor, typically below 5 percent, or the lifetime of long-lifetime luminescence is shorter than 1 microsecond, typically less than 100 nanoseconds. Examples of non-luminescent compounds are lanthanide chelates, which do not contain a light-harvesting antenna structure for efficient excitation of the lanthanide ion, and light-harvesting antenna ligands, which are not coordinated to lanthanide ions and, thus, not able to produce long lifetime luminescence.

The terms “lanthanide luminescence” and “luminescence” shall be understood to mean luminescence (i.e. light emission) obtained from emissive relaxation of electronic transitions of lanthanide ion. Lanthanide luminescence can be generated by excitation of the lanthanide ion by direct or indirect light absorption or by electrogenerated chemical excitation.

The term “chelate” is defined as a coordination complex where a single central metal ion is coordinated to at least one ligand with at least one coordination bond. These complexes may be named by different principles, and names like chelates, supramolecular compounds, complexes and complexones are used. Special types of chelates include e.g. polyaminocarboxylic acids, macrocyclic complexes, crown ethers, cryptates, calixarenes and phorphyrins. The term “mixed chelate” shall be understood as a chelate comprising at least two different ligands coordinated with at least one coordination bond each.

The terms “time-resolved lanthanide fluorescence”, “time-resolved fluorescence”, “long-lifetime lanthanide luminescence” and “long-lifetime fluorescence” shall be understood here as lanthanide luminescence, where a luminescence lifetime of the luminescent compound is equal to or more than 1 microsecond (the lifetime being calculated as the time wherein luminescence emission intensity decays to the relative value of 1/e, i.e. to approximately 37% of the original luminescence emission intensity). Examples of compounds capable of long-lifetime fluorescence include, but are not limited to, intrinsically fluorescent chelate complexes of europium(III), samarium(III), terbium(III) and dysprosium(III) containing appropriate light-harvesting antenna.

The terms “light”, “excitation light” and “emission light” shall be understood to cover electromagnetic radiation at wavelengths from 200 nm to 1600 nm. These wavelengths are called ultraviolet light below 400 nm, near-ultraviolet light between 300-450 nm, visible light between 400-750 nm, near-infrared light between 700-1000 nm and infrared light above 700 nm.

The terms “short-lifetime fluorescence” and “short-lifetime fluorescent compound” shall be understood to cover fluorescence and fluorescent compounds with a luminescence lifetime of less than 1 microsecond, preferably less than 100 nanoseconds. The short-lifetime fluorescence is also referred by conventional fluorescence.

The terms “electrogenerated luminescence” and “electrochemiluminescence” shall be understood here as lanthanide luminescence produced by electrogenerated chemical excitation using an electrode and applying electric current or voltage to the electrode. Depending on the electrode where the electrochemical reaction producing luminescence occurs the electrochemiluminescence is called cathodic or anodic electrochemiluminescence. Electrogenerated luminescence compounds are compounds capable of anodic or cathodic electrogenerated luminescence. An example of such a compound is hot electron excited 2,6-bis[N,N-bis(carboxymethyl)-aminomethyl]-4-benzoyl phenol-chelated Tb(III) producing green emission (J Alloys Comp 1995; 225: 502-506), but other lanthanide complexes capable of electrogenerated luminescence exist. Electrogenerated luminescence of lanthanide complexes can also be measured using temporal resolution to improve limit of detection.

In this disclosure, the term “bioassay” shall be understood to refer to detection and/or quantitation of analyte based on fluorescence or luminescence and utilizing probe pair where at least one of the probes contains double-stranded stem-loop structure. The analyte is typically detected and/or measured from a sample or an aliquot of sample, which sample is e.g. a biological or clinical sample..

The term “homogeneous bioassay” shall be understood to cover bioassays requiring no separation steps. Single or multiple steps of each; addition of reagents, incubation and measurement are the only steps required. The term “separation step” shall be understood to be a step where a labelled bioassay reagent bound onto a solid-phase, such as for example a microparticle or a microtitration well, is separated and physically isolated from the unbound labelled bioassay reagent; for example the microtitration well is washed (liquid is taken out and, to improve the separation, additional liquid is added and the well emptied) resulting in separation of the solid-phase bound labelled bioassay reagent from the labelled bioassay reagent not bound onto the solid-phase.

The terms “analyte” and “nucleic acid analyte” shall be understood herein as a polynucleotide substance of interest, which is to be measured by the bioassay from the sample. DNA (deoxyribonucleic acid) and RNA (ribonucleic acid) are examples of polynucleotides.

The terms “sample” and “biological sample” shall be understood to cover various liquid or solid biological samples whereof the analyte is detected, such as serum, blood, plasma, saliva, urine, faeces, seminal plasma, sweat, liquor, amniotic fluid, tissue homogenate, ascites, samples from environmental studies (water and soil samples) and industrial processes (process solutions). The sample can be also product of pre-treatment process of biological sample.

### Preferred Embodiments of the Invention

A typical luminescence hybridization assay method for detecting nucleic acid analyte and/or quantifying nucleic acid analyte concentration according to the invention employs a binary probe system, comprising a step of contacting two oligonucleotide probes with a sample, wherein
(a) a first oligonucleotide probe comprises a double-stranded terminal stem-loop structure and a single-stranded terminal overhang sequence that is complementary to and is capable of selectively hybridizing with a first region of the nucleic acid analyte, and
b) a second oligonucleotide probe comprises a single-stranded terminal sequence that is complementary to and is capable of selectively hybridizing with a second region of the nucleic acid analyte, and
said first region of the nucleic acid analyte is terminal region and said single-stranded terminal overhang sequence of said first oligonucleotide probe hybridizes to said first region of the nucleic acid analyte, and said hybridization results in formation of a first nick structure between a terminus of said nucleic acid analyte and a first terminus of said first oligonucleotide probe, and said first terminus of said first oligonucleotide probe is part of said double-stranded terminal stem-loop structure of said first oligonucleotide probe, wherein the first and second region of the nucleic acid analyte are adjacent regions of the nucleic acid analyte, and the single-stranded terminal sequences of the first and second oligonucleotide probe hybridize to the nucleic acid analyte forming a second nick structure between a second terminus of the first oligonucleotide probe and first terminus of the second oligonucleotide probe;

and detecting the presence or absence of said nucleic acid analyte bound to said first and second oligonucleotide probes, wherein the presence of said nucleic acid analyte bound to said first and second oligonucleotide probes confirms the presence of the analyte in the sample.

According to another embodiment of the invention also the second oligonucleotide probe comprises a double-stranded terminal stem-loop structure and a single-stranded terminal overhang sequence that is complementary to and is capable of selectively hybridizing with a second region of the nucleic acid analyte, and the second region of the nucleic acid analyte is terminal region and the single-stranded terminal overhang sequence of second oligonucleotide probe hybridizes to the second region of the nucleic acid analyte, and the hybridization results in formation of a third nick structure comprising first terminus of the nucleic acid analyte and second terminus of the second oligonucleotide probe comprising the double-stranded terminal stem-loop structure, and the second terminus of the second oligonucleotide probe is part of the double-stranded terminal stem-loop structure of the second oligonucleotide probe.

According to the invention the first terminuses are 3′ ends (three prime ends) and the second terminuses are 5′ ends (five prime ends), or the first terminuses are 5′ ends (five prime ends) and the second terminuses are 3′ ends (three prime ends).

In typical embodiments of the invention, the nucleic acid analyte is a polynucleotide biopolymer composed of nucleotide monomers, typically either deoxyribonucleotides or ribonucleotides, covalently bonded in a linear chain. In a linear polynucleotide chain, the sequence of nucleotides are linked by phosphodiester bonds present between the adjacent nucleotides, and the linear sequence of nucleotides comprises a 5′ terminal nucleotide, a 3′ terminal nucleotide and plurality of internal nucleotides between said terminal nucleotides. The linear polynucleotide chain can be present either in a linear conformation or twisted and/or folded into three-dimensional conformations stabilized by intramolecular noncovalent bonds that can be destabilized by increased temperature.

In typical embodiments of the invention the nucleic acid analyte is a single stranded nucleic acid with length of 10 - 50 nucleotides, more preferably 15 - 30 nucleotides and most preferably 16 - 26 nucleotides.

In typical embodiments of the invention the nucleic acid analyte is a microRNA with length of 17 - 25 nucleotides, and in most typical embodies microRNA with length of 21 - 22 nucleotides.

In preferred embodiments of the invention, the length of the single-stranded terminal overhang sequence of the stem-loop probe oligonucleotide is 5 - 25 nucleotides, more preferably 5 - 20 nucleotides, most preferably 5 - 15 nucleotides.

In preferred embodiments of the invention the length of double stranded stem sequence in the stem-loop structure is 4 - 16 base pairs, i.e. the palindromic sequence, located on both side of the loop, has length of 4 - 16 nucleotides.

In preferred embodiments of the invention the length of single stranded loop sequence in the double stranded stem-loop structure is 1 - 12 nucleotides, more preferably 3 - 8 nucleotides, most preferably 4 - 7 nucleotides.

In preferred embodiments of the invention the first and second oligonucleotide probes comprise DNA (deoxyribonucleic acids), RNA (ribonucleic acids) or their synthetic analogues such as LNA (locked nucleic acids) and PNA (peptide nucleic acid) or any combination of them, and in the most preferred embodiments of the invention the first and second oligonucleotide probes comprise only DNA or RNA nucleotides.

In typical embodiments of the invention the first and second oligonucleotide probes are both labelled, or both probes are labelled and one of the probes is further coupled to any kind of solid support. The coupling of the stem-loop probe to the solid-support is preferably via modified nucleotide in the the loop sequence. Examples of such immobilized stem-loop probes with single stranded overhang are described in Nucleic Acids Res. 2001; 29: e92 explaining the preferred length of the loop sequence as 7 nucleotides and comprising the NH₂-C6-dT nucleotide in the middle of the loop sequence.

According to one embodiment of the invention, the target nucleic acid analyte is detected and/or quantified by using agarose gel electrophoresis to separate the the complex formed upon hybridization of the first and second oligonucleotide probes with the analyte and visualizing the formed complex using intercalating dye. The complex formed has different mobility on the agarose gel electrophoresis compared to the individual probes or the analyte due to difference in size and conformation. An example of miRNA detection assay based on unlabeled probe hybridization reaction and agarose gel electrophoretic separation of hybridization complex is described in [Sciences Advances 2019; 5: aau9443].

According to yet another embodiment of the invention, the target nucleic acid analyte is detected and/or quantified by using melting curve analysis of the complex formed upon hybridization of the first and second oligonucleotide probes with the analyte and double stranded nucleic acid (dsDNA, dsRNA and DNA:RNA-hybrid) binding dye such as SYBR Green I. The dye binds and fluoresces upon binding to double stranded nucleic acid complexes and the melting curve analysis can be used to the confirm the presence of the complex formed with the analyte. An example of miRNA and nucleic acid sequence mutation detection based on unlabeled probe hybridization reaction and melting curve analysis with SYBR Green I is described in [The Analyst 2013: 141: 2384-2387, doi:10.1039/c6an00001k; PloS ONE 2011; 6: e26534].

In preferred embodiments of the invention the target nucleic acid analyte is detected and/or quantified using fluorescence measurement, more preferably using time-gated fluorescence measurement.

According to one embodiment of the invention the first and second oligonucleotide probes are labeled with a fluorescence resonance energy transfer pair, wherein, either
(a) the first oligonucleotide probe comprises a fluorescent donor and the second oligonucleotide probe comprises a fluorescent acceptor or quencher, or
(b) the first oligonucleotide probe comprising a fluorescent acceptor or quencher and the second oligonucleotide probe comprising a fluorescent donor; and
wherein resonance energy transfer between the fluorescence resonance energy transfer pair is enabled upon occurrence of both the hybridization events, hybridization of the single-stranded terminal sequence of the first oligonucleotide probe to the first region of the nucleic acid analyte, and hybridization the single-stranded terminal sequence of the second oligonucleotide probe to the second region of the nucleic acid analyte.

According to one embodiment of the invention, when sensitized acceptor emission is measured, the donor and the acceptor are attached during labeling to such positions in the oligonucleotide probes that after hybridization to the nucleic acid analyte the positions of the label carrying nucleotides are preferably separated by at least 4 nucleotides, more preferably by at least 8 nucleotides, but no more than 24 nucleotides.

The fluorescence resonance energy donor is preferably a luminescent lanthanide chelate comprising a lanthanide ion. Examples of suitable lanthanide donors are Lumi4-Tb cryptate, Eu-TBP (trisbipyridine) cryptate, Eu-W1024 chelate, Eu-W1284 and Eu-W8044 chelates, as well as nonadentate and decadentate Eu(III) chelates described Anal Chem (2003) 75:3193-201 and Inorg Chem. (2013) 52:8461-6.

The fluorescent acceptor can be selected based on the spectral overlap between the donor emission and acceptor excitation spectrum from a large variety of fluorescent dyes, preferably from short-lifetime fluorescent compounds with excitation and emission at visible or near-infared region of the spectrum (such as ATTO, QXL, Alexa, Bodipy and Cyanine dyes). Examples of suitable fluorescent acceptors to be used in combination with fluorescent Eu(III) or Tb(III) chelate as donor are ATTO 647 and 647N, Alexa Fluor® 647 and 647N, Cy 3, Cy 3.5, Cy 5, Cy 5.5, Cy 7, XL 665 (crosslinked allophycocyanin), Chromeo™ 494, ATTO 490LS, QXL 610, QXL 670, QXL 680, LC red, Quasar 670, and Oyster 645.

The non-fluorescent quencher is preferably selected from universal quenchers not requiring efficient spectral overlap such as Dabcyl or any of BHQ or QSY quenchers. Examples of suitable non-fluorecent acceptors to be used in combination with fluorescent Eu(III) or Tb(III) chelate as donor are are dabcyl (dimethylaminoazobenzenesulfonic acid), Iowa Black RQ, IRDye QC-1, BHQ-0, BHQ-1, BHQ-2, BHQ-3, QSY 21 and DDQ-II.

According to another embodiment of the invention the first and second oligonucleotide probes are labelled with a switchable lanthanide luminescence label system, wherein, either
(a) the first oligonucleotide probe comprises a lanthanide ion carrier ligand and a lanthanide ion and the second oligonucleotide probe comprises an antenna ligand, or
(a) the first oligonucleotide probe comprises an antenna ligand and the second oligonucleotide probe comprises a lanthanide ion carrier ligand and a lanthanide ion; and
wherein luminescence of the switchable lanthanide luminescence label system is switched on upon occurrence of both the hybridization events, hybridization of the single-stranded terminal sequence of the first oligonucleotide probe to the first region of the nucleic acid analyte, and hybridization of the single-stranded terminal sequence of the second oligonucleotide probe to the second region of the nucleic acid analyte.

According to one embodiment of the invention, when the two oligonucleotide probes hybridize to adjacent regions in the nucleic acid analyte, the lanthanide ion carrier chelate and the antenna ligand are attached during labeling to such positions in the oligonucleotide probes that after hybridization to the nucleic acid analyte the positions of the label carrying nucleotides are separated by at least one nucleotide, preferably by 2 - 4 nucleotides to sterically favor the self-assembly and formation of the mixed chelate complex.

The antenna ligand preferably binds weakly to said lanthanide ion, i.e. in typical embodiments of the invention the antenna ligand is either monodentate, bidentate, tridentate or tetradentate. Examples of preferred antenna ligands such as 4-((4-isothiocyanatophenyl)ethynyl)pyridine-2,6-dicarboxylic acid and 4-((4-((4,6-dichloro-1,3,5-triazin-2-yl)amino)phenyl)ethynyl)pyridine-2,6-dicarboxylic acid are described in PCT Int. Appl. WO 2010/109065 and Analyst 2017; 142:2411-2418, respectively.

In typical embodiments of the invention the ion carrier chelate is pentadentate, hexadentate, heptadentate or octadentate, preferably hexadentate, heptadentate or octadentate. e.g. the lanthanide ion carrier ligand is derived from linear or cyclic chelators, such as EDTA and DTPA or NOTA and DOTA, respectively. Examples of preferred ion carrier ligands such as (2,2′,2″-(10-(3-isothiocyanatobenzyl)-1,4,7,10-tetraazacyclododecane-1,4,7-triyl)triacetic acid and N1-(4-isothiocyanatobenzyl)diethylenetriamine-N¹,N²,N³,N³-tetraacetate) are described in PCT Int. Appl. WO 2010/109065.

The lanthanide ion is preferably selected from the group consisting of praseodymium(III), neodymium(III), samarium(III), europium(III), terbium(III), dysprosium(III), holmium(III), erbium(III), thulium(III) and ytterbium(III).

According to a yet another embodiment of the invention, more than one different binary probe system, each composed of at least partially different the first and/or second oligonucleotide probes, is used to construct a multiparametric assay and to detect simultaneously multiple different short nucleic acid analytes from the same sample. Typically, each of the different binary probe systems comprises a different lanthanide ion and/or one of the probes of each binary probe system is coupled to spatially separated surface area on any kind of solid support.

In preferred embodiments of the invention the emission of the fluorescent complex formed, the emission of the fluorescent donor or the sensitized emission of the fluorescent acceptor emission is measured at a wavelength between 400 and 1600 nm.

In typical embodiments of the invention upon presence of the target nucleic acid the fluorescence measured of the fluorescent complex formed or the sensitized emission of the acceptor is increased, while the fluorescence measured of the fluorescent donor is decreased, and in preferred embodiments of the invention the presence of the target nucleic acid can be detected and quantified without any separation step.

According to one embodiment of the invention, when one of the probes is coupled to any kind of solid support, a separation step can be included and only the solid-phase bound signal is read to detect and quantify the target nucleic acid.

In preferred embodiments of the invention the fluorescence is measured with time-gating, i.e. only the delayed fluorescence is observed. Typical time-gating with lanthanide luminescence comprises a delay of at least 10 µs between the end of excitation and start of emission measurement.

According to one embodiment of the invention the 5′ hydroxyl terminuses of the oligonucleotide probes are non-phosphorylated.

According to another embodiment of the invention, the 5′ hydroxyl terminus of at least one of oligonucleotide probes is phosphorylated and a ligase enzyme is used to form a covalent bond in a place of any one or any combination of the first, second and third nick structures formed upon occurrence of any of the hybridization events between the first oligonucleotide probe, said second oligonucleotide probe and the nucleic acid analyte. For example T4 RNA ligase 2 is able to catalyze ligation of DNA-fragments on RNA template and some DNA ligases can also efficient join of 3′-OH-terminated RNA to 5′-phosphate-terminated DNA on a DNA template [Biochemistry 1997; 36: 9073-9079.]

The ligase enzyme treatment is preferably done at lower temperature than the fluorescence measurement to destabilize and preferably dissociate the hybridized probes in case the nicks area not ligated.

According to yet another embodiment of the invention, the luminescence hybridization assay based on a binary probe system, comprising two oligonucleotide probes, wherein at least one of the probes comprises a stem-loop structure on a binary probe system, is used in in situ hybridization.

According to yet another embodiment of the invention, the oligonucleotide probe hybridization is carried out in temperature between 15 - 80° C., more preferably between 15 - 60° C. and most preferably between 20 - 40° C. The measurement is done at the same or at different temperature than the oligonucleotide probe hybridization.

According to yet another embodiment of the invention the oligonucleotide probe hybridization is carried out in presence of 150 mM - 1 M concentration of NaCl. The measurement is done at the same or at different concentration of NaCl than the oligonucleotide probe hybridization.

According to one embodiment of the invention, the nucleic acid analyte is detected or quantified from body fluids such as plasma and serum. In preferred embodiments of the invention no enzymatic nucleic amplification or nucleic acid extension is used, but the pretreatment of the sample can include e.g. immunocapture or other nucleic acid analyte concentrating step.

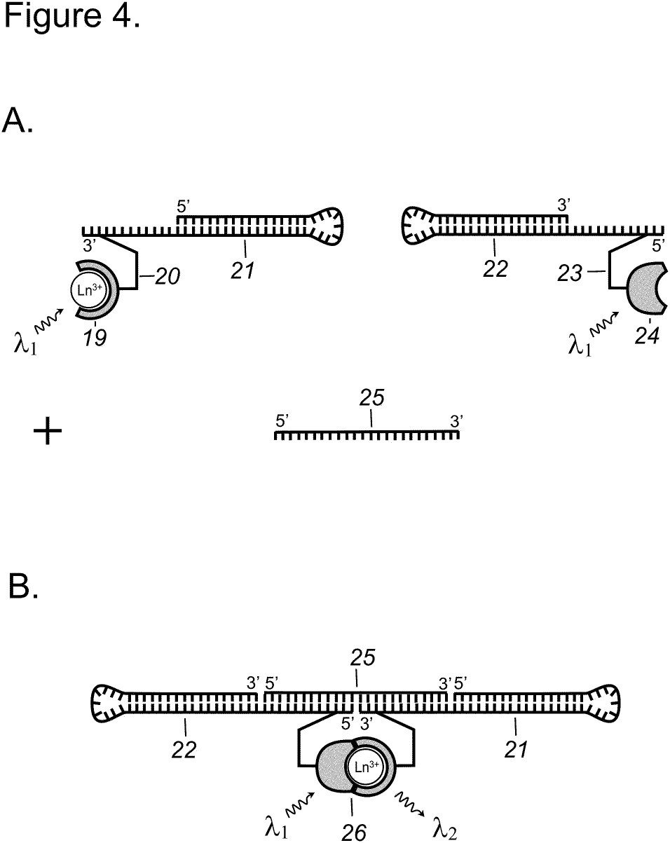

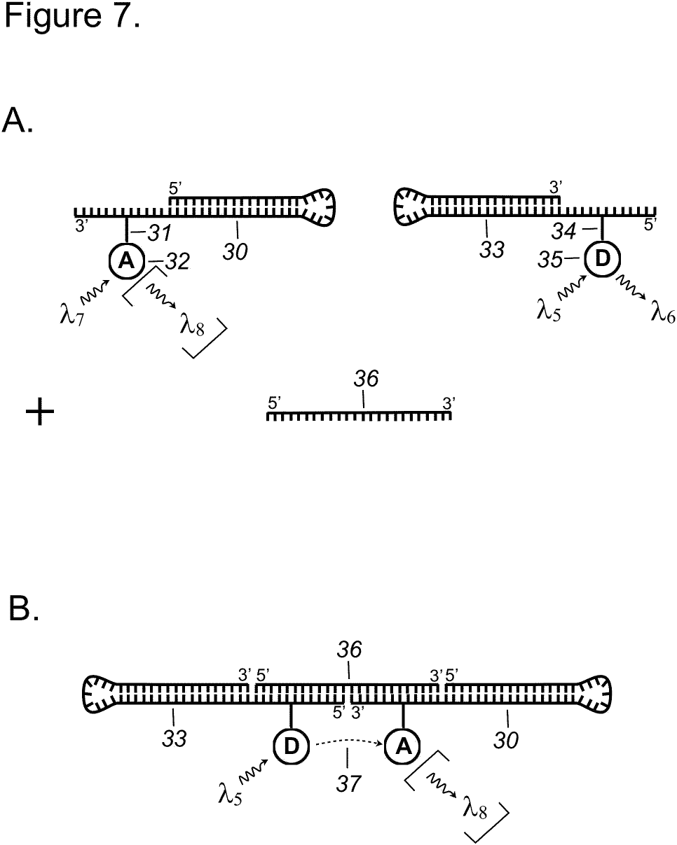

Examples of alternative embodiments of the invention, the luminescence hybridization assay based on a binary probe system, comprising two oligonucleotide probes, wherein at least one of the probes comprises a stem-loop structure and detection and quantitation of the target nucleic acid requires simultaneous binding of the two oligonucleotide probes to the same nucleic acid target molecule, are further described in FIGS. 4 - 8. FIGS. 4 - 6 and 8 describe alternative embodiments of the invention utilizing chelate complementation based detection and FIG. 7 describes a fluorescence resonance energy transfer based embodiment of the invention.

FIG. 4 illustrates an example of a luminescence binary probe hybridization assay system, comprising two chelate-complementation oligonucleotide probes, wherein a first oligonucleotide probe (21), labeled with a lanthanide ion carrier chelate (19) via a linker (20), comprises a double-stranded 5′ terminal stem-loop sequence and a single-stranded 3′ overhang, that is complementary to the 3′ terminal region of the nucleic acid analyte (25); and wherein a second oligonucleotide probe (22), labeled with a light-harvesting antenna (24) via a linker (23), comprises a double-stranded 3′ terminal stem-loop sequence and a single-stranded 5′ overhang that is complementary to the 5′ terminal region of the nucleic acid analyte (25). The regions of the nucleic acid analyte that are complementary to the probe sequences are located adjacently in the sequence of the nucleic acid analyte. Upon excitation at lambda-1 wavelength the lanthanide ion carrier chelate (21) and the light-harvesting antenna (24) are practically non-luminescent, when the probes are not bound to the same nucleic acid analyte molecule. (B) Both first (21) and second (22) oligonucleotide probe hybridize to their complementary regions of the nucleic acid analyte molecule (25). The adjacent hybridization of the probes results in directed self-assembly and formation of the fluorescent chelate complex (26), which produces luminescence emission at lambda-2 wavelength upon excitation at lambda-1 wavelength.

The hybridization of the two stem-loop oligonucleotide probes on the described adjacent regions on the target nucleic acid analyte results in formation of three stabilizing nick structures that enable strong co-operative binding of the two probes even they possess only very short complementary sequences. The co-operative binding is further enhanced by the coordination of the light-harvesting antenna ligand to the lanthanide ion in the ion carrier chelate. In absence of any of the factors affecting this co-operative stabilization effect the dissociation of the probes is favored. The highly selective hybridization renders the formation of the fluorescent chelate complex strictly specific to the presence of the defined 3′ and 5′ terminal sequences in and the defined length of the nucleic acid analyte. In case any difference in the terminal sequences exists or the length of the sequence of the nucleic acid analyte does not match the defined length, i.e. the nucleic acid analyte has either deletion of nucleotides in the sequence or comprises a terminal overhang, at least one of the probes is stays dissociated and thus the fluorescence chelate complex is not formed. The specificity of the method to detect and quantify short nucleic acid analytes is thus unique, and the cooperative stabilization effect enables detection of shorter nucleic acid analyte than possible with existing hybridization probe assays or binary probe hybridization assays.

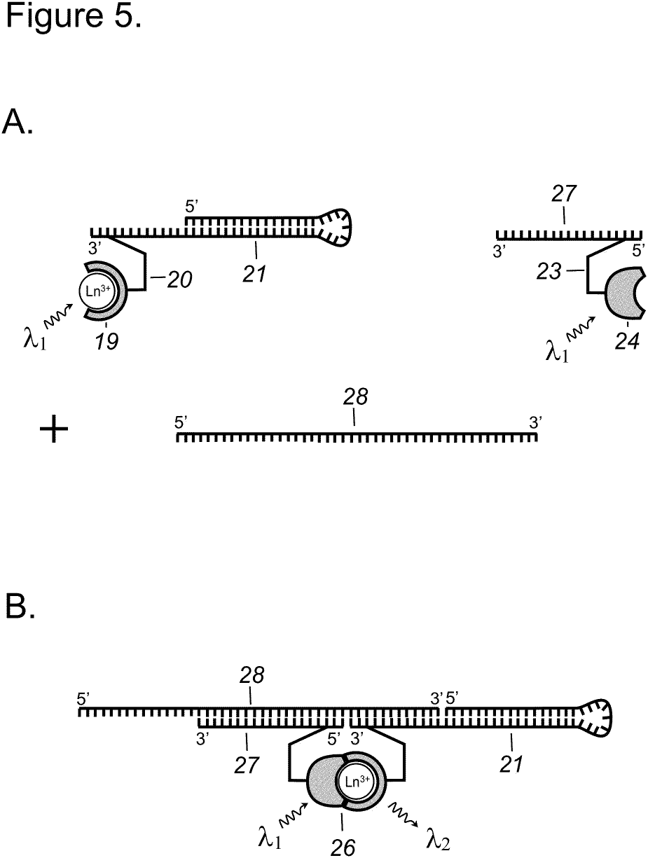

FIG. 5 illustrates an example of a luminescence binary probe hybridization assay system, comprising two chelate-complementation oligonucleotide probes, wherein a first oligonucleotide probe (21), labeled with a lanthanide ion carrier chelate (19) via a linker (20), comprises a double-stranded 5′ terminal stem-loop sequence and a single-stranded 3′ overhang, that is complementary to the 3′ terminal region of the nucleic acid analyte (28); and wherein a second oligonucleotide probe (27), labeled with a light-harvesting antenna (24) via a linker (23), comprises a linear single-stranded oligonucleotide complementary to the nucleic acid analyte (25) non-overlapping with the 3′ terminal region, that is complementary to the first oligonucleotide probe. The regions of the nucleic acid analyte that are complementary to the probe sequences are located adjacently in the sequence of the nucleic acid analyte. Upon excitation at lambda-1 the lanthanide ion carrier chelate (21) and the light-harvesting antenna (24) are practically non-luminescent when the probes are not bound to the same nucleic acid analyte molecule. (B) Both first (21) and second (27) oligonucleotide probe hybridize to their complementary regions of the nucleic acid analyte molecule (28). The adjacent hybridization of the probes results in directed self-assembly and formation of the fluorescent chelate complex (26), which produces luminescence emission at lambda-2 wavelength upon excitation at lambda-1 wavelength.

The hybridization of the stem-loop oligonucleotide probe and the linear oligonucleotide probe on the adjacent regions on the nucleic acid analyte results in formation of two stabilizing nick structures that enable strong co-operative binding of the two probes even especially the stem-loop probe possess only very short complementary sequence. In absence of this co-operative stabilization effect the dissociation of the probes is favored. The highly selective hybridization renders the formation of the fluorescent chelate complex strictly specific to the presence of the defined 3′ terminal sequence in the nucleic acid analyte.

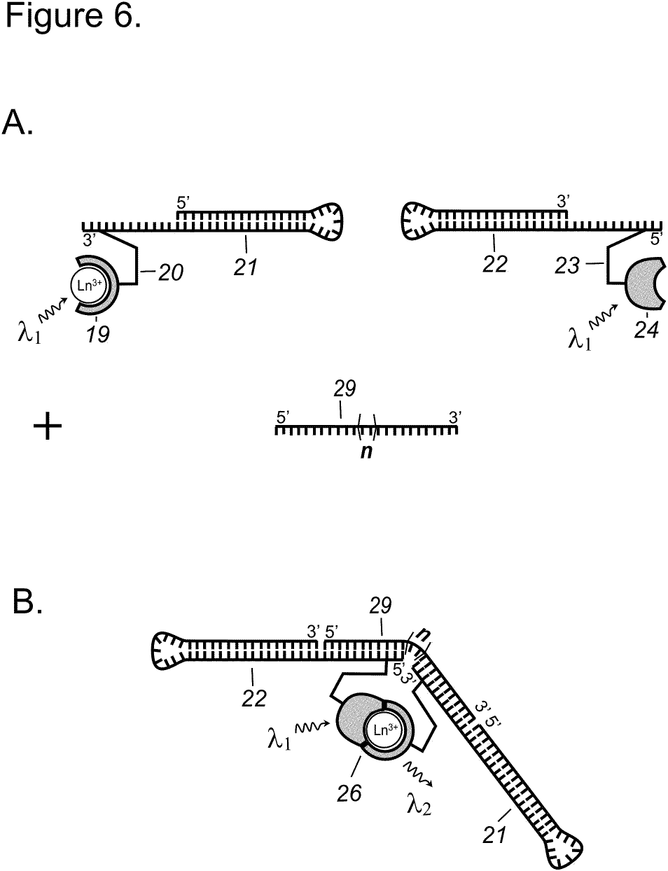

FIG. 6 illustrates an example of a luminescence binary probe hybridization assay system, comprising two chelate-complementation oligonucleotide probes, wherein a first oligonucleotide probe (21), labeled with a lanthanide ion carrier chelate (19) via a linker (20), comprises a double-stranded 5′ terminal stem-loop sequence and a single-stranded 3′ overhang, that is complementary to the 3′ terminal region of the nucleic acid analyte (29); and wherein a second oligonucleotide probe (22), labeled with a light-harvesting antenna (24) via a linker (23), comprises a double-stranded 3′ terminal stem-loop sequence and a single-stranded 5′ overhang that is complementary to the 5′ terminal region of the nucleic acid analyte (29). In the sequence of the nucleic acid analyte the regions that are complementary to the probe sequences are separated by one or multiple non-defined intermediate nucleotides (n) composing a short non-recognized part of the sequence. Upon excitation at lambda-1 wavelength the lanthanide ion carrier chelate (21) and the light-harvesting antenna (24) are practically non-luminescent, when the probes are not bound to the same nucleic acid analyte molecule. (B) Both first (21) and second (22) oligonucleotide probe hybridize to their complementary regions of the nucleic acid analyte (29). The hybridization of the probes to the nucleic acid analyte results in bending of the nucleic acid analyte molecule and directed self-assembly and formation of the fluorescent chelate complex (26), which produces luminescence emission at lambda-2 wavelength upon excitation at lambda-1 wavelength.

The hybridization of the two stem-loop oligonucleotide probes on the described adjacent regions on the target nucleic acid analyte results in formation of two stabilizing nick structures that enable strong co-operative binding of the two probes even the probes possess only very short complementary sequences. The co-operative binding is further enhanced by the coordination of the light-harvesting antenna ligand to the lanthanide ion in the ion carrier chelate. In absence of this co-operative stabilization effect the dissociation of the probes is favored.

FIG. 7 illustrates an example of a luminescence binary probe hybridization assay system, comprising two oligonucleotide FRET probes, wherein a first oligonucleotide probe (30), labeled with a fluorescent acceptor (32) via a linker (31), comprises a double-stranded 5′ terminal stem-loop sequence and a single-stranded 3′ overhang, that is complementary to the 3′ terminal region of the nucleic acid analyte (36); and wherein a second oligonucleotide probe (33), labeled with a fluorescent donor (35) via a linker (34), comprises a double-stranded 3′ terminal stem-loop sequence and a single-stranded 5′ overhang that is complementary to the 5′ terminal region of the nucleic acid analyte (36). The fluorescent donor and the fluorescent acceptor form a FRET-pair. The regions of the nucleic acid analyte that are complementary to the probe sequences are located adjacently in the sequence of the nucleic acid analyte. Upon excitation at lambda-5 wavelength the fluorescent acceptor (32) and the fluorescent acceptor (35) are practically non-luminescent at lambda-8 wavelength, when the probes are not bound to the same nucleic acid analyte molecule. (B) Both first (30) and second (33) oligonucleotide probe hybridize to their complementary regions of the nucleic acid analyte molecule (36). The adjacent hybridization of the probes results in proximity of the fluorescent donor and fluorescent acceptor and enable non-radiative energy-transfer (37), which produces luminescence emission at lambda-8 wavelength upon excitation at lambda-5 wavelength. The non-radiative energy-transfer (37) enabled by the proximity of the fluorescent donor and the acceptor can be observed also in case of non-luminescent acceptor by decrease of luminescence emission at lambda-6 upon excitation of lambda-5.

The hybridization of the two stem-loop oligonucleotide probes on the described adjacent regions on the target nucleic acid analyte results in formation of three stabilizing nick structures and provides highly selective and sensitive detection of the target nucleic acid equally as in FIG. 4.

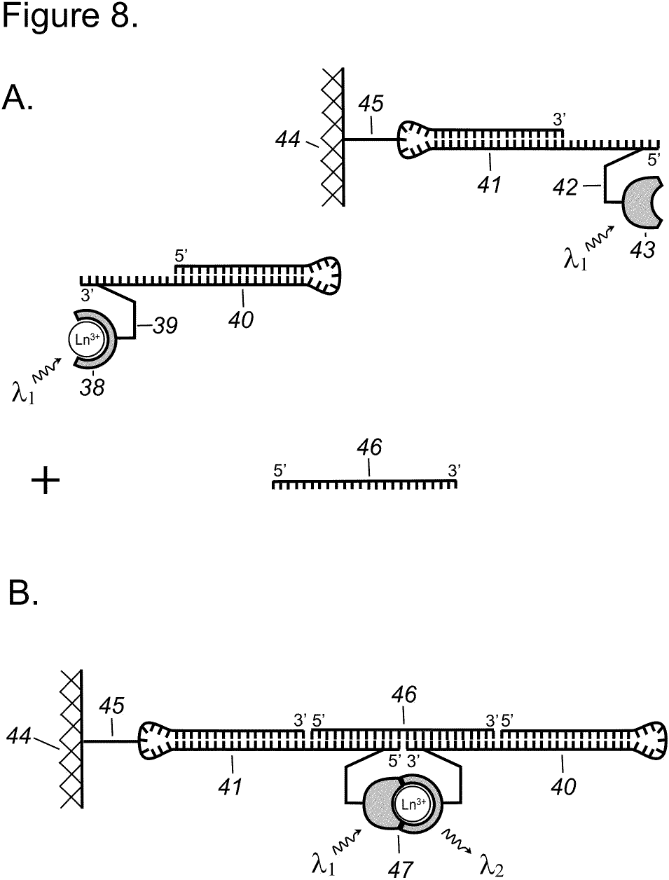

FIG. 8 illustrates an example of a luminescence binary probe hybridization assay system on solid support, comprising two oligonucleotide probes, wherein a first oligonucleotide probe (40), labeled with a lanthanide ion carrier chelate (38) via a linker (39), comprises a double-stranded 5′ terminal stem-loop sequence and a single-stranded 3′ overhang, that is complementary to the 3′ terminal region of the nucleic acid analyte (46); and wherein a second oligonucleotide probe (41), labeled with a light-harvesting antenna (43) via a linker (42) and coupled to solid support (44) via another linker (45), comprises a double-stranded 3′ terminal stem-loop sequence and a single-stranded 5′ overhang that is complementary to the 5′ terminal region of the nucleic acid analyte (46). The regions of the nucleic acid analyte that are complementary to the probe sequences are located adjacently in the sequence of the nucleic acid analyte. Upon excitation at lambda-1 wavelength the lanthanide ion carrier chelate (21) and the light-harvesting antenna (24) are practically non-luminescent, when the probes are not bound to the same nucleic acid analyte molecule. (B) Both first (40) and second (41) oligonucleotide probe hybridize to their complementary regions of the nucleic acid analyte molecule (46). The adjacent hybridization of the probes results in directed self-assembly and formation of the fluorescent chelate complex (47), which produces luminescence emission at lambda-2 wavelength upon excitation at lambda-1 wavelength. The assay can comprise a separation step (wash step) to remove the components not bound on the surface of the solid support before the luminescence measurement. The solid-support can further comprise multiple spatially separated areas each coupled to a different binary probe hybridization assay system each recognizing a different nucleic acid analyte.

The hybridization of the two stem-loop oligonucleotide probes on the described adjacent regions on the target nucleic acid analyte results in formation of three stabilizing nick structures and provides highly selective and sensitive detection of the target nucleic acid equally as in FIGS. 4 and 7.

A further embodiment provided by the present invention is a kit for detecting and/or quantitating a short nucleic acid analyte molecule, the kit comprising:
(a) a first oligonucleotide probe comprising a double-stranded terminal stem-loop structure and a single-stranded terminal sequence overhang that is complementary to and is capable of selectively hybridizing with a first region of a single-stranded nucleic acid analyte, and
b) a second oligonucleotide probe comprising a double-stranded terminal stem-loop structure and a single-stranded terminal sequence overhang that is complementary to and is capable of selectively hybridizing with a second region of said single-stranded nucleic acid analyte,

wherein said first and second region of the single-stranded nucleic acid analyte are strictly adjacent regions of said single-stranded nucleic acid analyte,
wherein a terminal nucleotide in a terminus of the single-stranded terminal sequence overhang of said first oligonucleotide probe and a terminal nucleotide in a terminus of the single-stranded terminal sequence overhang of said second oligonucleotide probe are designed to bind to adjacent nucleotides in said single-stranded nucleic acid analyte in order to form a first nick structure between said terminal nucleotides of the single-stranded terminal sequence overhangs of said first and second oligonucleotide probes when said probes are bound to said single-stranded nucleic acid analyte, said first nick structure lacking a phosphodiester bond between a pair of adjacent nucleotides bound to a complementary strand; and
wherein said single-stranded terminal sequence overhangs of said first and second oligonucleotide probes are further designed so that when said probes are bound to said single-stranded nucleic acid analyte, one of the terminal nucleotides of said single-stranded nucleic acid analyte forms a second nick structure with a terminal nucleotide in a terminus which is part of said double-stranded terminal stem-loop structure of the first oligonucleotide probe and another of the terminal nucleotides of said single-stranded nucleic acid analyte forms a third nick structure with a terminal nucleotide in a terminus which is part of said double-stranded terminal stem-loop structure of the second oligonucleotide probe, said second and third nick structures lacking a phosphodiester bond between a pair of adjacent nucleotides bound to a complementary strand.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

### EXAMPLES

### Example 1

### Luminescent Hybridization Assay Using Binary Stem-Loop Oligonucleotide Probe System and Chelate Complementation

Synthetic target-1 DNA oligonucleotide (5′-TAAAGTGCTTATAGTGCAGGTAG-3′; SEQ ID NO: 1), target-2 DNA oligonucleotide (5′-CAAAGTGCTCATAGTGCAG GTAG-3′; SEQ ID NO: 2), target-3 DNA oligonucleotide (5′-CTTAAAGTGCTTATA GTGCAGGTAGAG-3′; SEQ ID NO: 3), target-4 DNA oligonucleotide (5′-AAGTGCTTATAGTGCAGGT-3′; SEQ ID NO: 4), amino-modified probe-C1 oligonucleotide (5′-GTGCTGACCGTAGTACCGGTCAGCACCTACCTGCA(NH₂-C6dC)-3′; SEQ ID NO: 5) and amino-modified probe-C2 oligonucleotide (5′-TA(NH₂-C6dT)AAGCACTTTAGCGTGCAGCCATACTAGGCTGCACGC-3′; SEQ ID NO: 6) were purchased from GeneLink (www.qenelink.com; Hawthorne, NY, USA).

Probe-C1 oligonucleotide was labelled with non-luminescent Eu³⁺ ion carrier chelate ((2,2′,2″-(10-(3-isothiocyanatobenzyl)-1,4,7,10-tetraazacyclododecane-1,4,7-triyl)tri(acetate) europium(III); DOTA-Eu (III)) [Analyst (2015) 140: 3960-3968] at the primary amino group residue of modified cytosine with six carbon linker, and probe-C2 oligonucleotide was labelled with light harvesting antenna ligand (4-((isothiocyanatophenyl)ethynyl)pyridine-2,6-dicarboxylic acid; 3d-antenna) [Analyst (2015) 140: 3960-3968] at the primary amino group residue of modified thymine with six carbon linker. Probe-C1 (25 nmol) was incubated with 20-fold molar excess of DOTA-Eu(III) in 50 mM carbonate buffer, pH 9.8, at +37° C. overnight. The reaction volume was 50 µL. For labelling of probe-C2 with 3d-antenna, the 3d-antenna was first dissolved in N,N-dimethylformamide, and probe-C2, 25 nmol, was incubated with 50-fold molar excess of 3d-antenna in 50 mM carbonate buffer, pH 9.8, at +50° C. overnight with slow rotation. The total reaction volume was 110 µL. The purification of labelled probes was carried out with HPLC (instrumentation from SpectraSystem, Thermo Fisher Scientific, Waltham, MA, USA) using a reverse-phase ODS C18 Hypersil column (150 mm long and i.d. of 4.6 mm) from Thermo Fisher Scientific. Purifications were performed using a linear acetonitrile gradient (from 15% to 40% acetonitrile in 30 min) in an aqueous 50 mM triethylammonium acetate buffer pH 7.0 with a flow rate of 0.5 mL min⁻¹. The collected fractions were evaporated and dried in miVac concentrator (GeneVac, Ipswich, UK) and dissolved again in storage buffer containing 10 mM Tris (pH 7.5), 50 mM NaCl and 10 µM EDTA. Labelled probes were characterized by measuring absorbance readings at 260 nm and 330 nm. Eu³⁺ concentration and labeling degree of probe-C1 was measured with DELFIA technology (PerkinElmer Life and Analytical Sciences, Wallac, Turku, Finland).

The luminescence hybridization assay was performed by using low Fluorescence 96-well Maxisorp microtitration plates purchased from Nunc (Roskilde, Denmark) in assay buffer containing 50 mM Tris-HCl (pH 7.75), 600 mM NaCl, 0.1% Tween 20, 0.05% NaN₃, and 30 µM diethylenetriaminepentaacetic acid (DTPA). The probe-C1 and probe-C2 were first denatured separately in 0.2 mL tubes by heating for 2 min at 98° C. and then rapidly cooled in 1 min to room temperature using PTC-200 DNA-Engine (MJ Research, Waltham, MA). The probe-C1 (20 nM) and probe-C2 (20 nM) the target-1 oligonucleotide (0-20 nM) were combined in a total volume of 60 µL and added to the microtitration wells. In addition, in separate control experiments target-1 was replaced with target-2 and target-3 oligonucleotides (20 nM). The plate was incubated first at slow shaking for a short period of time and then without shaking for 15 and 60 minutes at room temperature. Time-resolved fluorescence measurements were made with a 1420 Victor Multilabel Counter (Perkin-Elmer Life And Analytical Life Sciences) by using a 340 nm excitation filter, 615 nm emission filter (with 8 nm fwhm, full-width-at-half-maximum), 400 µs delay and 400 µs measurement time, and counting 1000 measurement cycles.

The experiment resulted in increasing time-resolved luminescence signal at 615 nm with increasing concentration of target-1 with limit of detection (3×SD) below 100 pM concentration, while target-2, target-3 or target-4 at 20 nM concentration produced no significant difference compared to background signal. This illustrated, that the assay is highly selective to target nucleic acid sequence composition as already few mismatches (target-2) disrupt the signal, and also to the exact length of the target nucleic acid is required, as no signal was generated when the target nucleic acid with longer (target-3) or shorter length (target-4) was tested. The hybridization of the probe pair requires thus, that the nick structures are formed upon probe hybridization at the both termini of the target nucleic acid and the base stacking effect is present. The luminescence hybridization assay according to the invention is thus highly selective: it is strongly dependent on the sequence of the target nucleic acid at both terminuses and the length of the target nucleic acid and any variation in length and/or sequence composition will disrupt the hybridization and and signal generation via chelate complementation.

### Example 2

### Luminescent Hybridization Assay Using Binary Stem-Loop Oligonucleotide Probe System and Fluorescence Resonance Energy Transfer

Synthetic target-1 DNA oligonucleotide (5′-TAAAGTGCTTATAGTGCAGGTAG-3′; SEQ ID NO: 1), target-2 DNA oligonucleotide (5′-CAAAGTGCTCATAGTGCAG GTAG-3′; SEQ ID NO: 2), target-3 DNA oligonucleotide (5′-CTTAAAGTGCTTATA GTGCAGGTAGAG-3′; SEQ ID NO: 3), target-4 DNA oligonucleotide (5′-AAGTGCTTATAGTGCAGGT-3′; SEQ ID NO: 4), amino-modified probe-F1 oligonucleotide (5′-GTGCTGACCGTAGTACCGGTCAGCACCTA(NH₂-C6dC)CT GCAC-3′; SEQ ID NO: 5) and amino-modified probe-F2 oligonucleotide (5′-TATAAG(NH₂-C6dC)ACTTTAGCGTGCAGCCATACTAGGCTGCACGC-3′; SEQ ID NO: 6) were purchased from GeneLink (www.genelink.com).

Probe-F1 oligonucleotide was labelled with intrinsically luminescent Eu³⁺ chelate, { 2,2′,2″,2‴-{[4-[(4-iso-thiocyanatophenyl)ethynyl]pyridine-2,6-diyl]-bis(methylene nitrilo)}-tetrakis(acetato)}europium(III) (7d-Eu) [J. Phys. Chem. B (2011) 115: 13685-13694] at the primary amino group residue of modified cytosine with six carbon linker, and probe-F2 oligonucleotide was labelled with Alexa Fluor 680 (succinimidyl ester dye; AF680; Thermo Fisher Scientific) at the primary amino group residue of modified cytosine with six carbon linker. Probe-F1 (5 nmol) and 60-fold molar excess of 7d-Eu were dissolved in a total volume of 30 µL of 50 mM carbonate buffer pH 9.8. The labeling reaction was incubated overnight at +37° C. protected from light. Probe-F2 (5 nmol) was dissolved with a 10-fold molar excess of the AF680 into 50 µL of 100 mM carbonate buffer pH 9.2. The labeling reaction was incubated overnight at +37° C. in a 6 rpm rotation protected from light. The labeled probe oligonucleotides were purified with reverse-phase HPLC (SpectraSYSTEM, Thermo Fisher Scientific) with reverse-phase ODS C18 Hypersil column (150 mm long and i.d. of 4.6 mm) from Thermo Fisher Scientific using a linear acetonitrile gradient in an aqueous 50 mM triethylammonium acetate buffer pH 7.0 and flow rate of 0.5 mL min⁻¹. The collected fractions were evaporated and dried in miVac concentrator (GeneVac) and dissolved again in storage buffer containing 10 mM Tris (pH 8.0), 50 mM NaCl and 10 µM EDTA. Labelled probes were characterized by measuring absorbance readings at 260 nm, 330 nm and 680 nm. Eu³⁺ concentration and labeling degree of probe-F1 was measured with DELFIA technology (PerkinElmer Life and Analytical Sciences, Wallac, Turku, Finland).

The luminescence hybridization assay was performed by using low Fluorescence 96-well Maxisorp microtitration plates purchased from Nunc in assay buffer containing 50 mM Tris-HCl (pH 7.75), 600 mM NaCl, 0.1% Tween 20, and 0.05% NaN₃. The probe-F1 and probe-F2 were first denatured separately in 0.2 mL tubes by heating for 2 min at 98° C. and then rapidly cooled in 1 min to room temperature using PTC-200 DNA-Engine (MJ Research, Waltham, MA). The probe-C1 (20 nM) and probe-C2 (20 nM) the target-1 oligonucleotide (0-20 nM) were combined in a total volume of 60 µL and added to the microtitration wells. In addition, in separate control experiments target-1 was replaced with target-2 and target-3 oligonucleotides (20 nM). The plate was incubated first at slow shaking for a short period of time and then without shaking for 15 and 60 minutes at room temperature. Time-resolved fluorescence measurements were made with a 1420 Victor Multilabel Counter (Perkin-Elmer Life And Analytical Life Sciences) by using a 340 nm excitation filter, 730 nm emission filter (with 10 nm fwhm), 60 µs delay and 80 µs measurement time, and counting 1000 measurement cycles.

The experiment resulted in increasing time-resolved luminescence signal at 730 nm with increasing concentration of target-1 with limit of detection (3×SD) below 200 pM concentration, while target-2, target-3 or target-4 at 20 nM concentration produced no significant difference compared to background signal. The luminescence hybridization assay based on binary stem-loop FRET-probe pair shows thus similar advantages than the equivalent assay based on chelate complementation probe pair. This indicates that the luminescence hybridization assay according to the present invention is highly specific to the target sequence and length, and is able to quantitatively detect small concentration of short target nucleic acid molecules that are otherwise hard to measure.

### OTHER PREFERRED EMBODIMENTS

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A bioassay method for detecting and/or quantitating a nucleic acid
(I) contacting two oligonucleotide probes with a sample, wherein
(a) a first oligonucleotide probe comprises a double-stranded terminal stem-loop structure and a single-stranded terminal sequence overhang that is complementary to and is capable of selectively hybridizing with a first region of the nucleic acid analyte, and
b) a second oligonucleotide probe comprises a single-stranded terminal sequence that is complementary to and is capable of selectively hybridizing with a second region of the nucleic acid analyte, wherein
said first region of the nucleic acid analyte is a first terminal region and said single-stranded terminal sequence overhang of said first oligonucleotide probe hybridizes to said first region of the nucleic acid analyte,
hybridization results in formation of a first nick structure between a terminus of said nucleic acid analyte and a first terminus of said first oligonucleotide probe, and
said first terminus of said first oligonucleotide probe is part of said double-stranded terminal stem-loop structure of said first oligonucleotide probe,
and wherein said first and second region of the nucleic acid analyte are strictly adjacent regions of said nucleic acid analyte, and said single-stranded terminal sequences of said first and second oligonucleotide probe hybridize to said nucleic acid analyte forming a second nick structure between a second terminus of said first oligonucleotide probe and a first terminus of said second oligonucleotide probe; and
(II) detecting the presence or absence of said nucleic acid analyte bound to said first and second oligonucleotide probes, wherein the presence of said nucleic acid analyte bound to said first and second oligonucleotide probes confirms the presence of the analyte in the sample.

2. The bioassay method according to claim 1 , wherein said second oligonucleotide probe comprises a double-stranded terminal stem-loop structure and said second region of the nucleic acid analyte is a second terminal region and said single-stranded terminal sequence of second oligonucleotide probe hybridizes to said second region of the nucleic acid analyte, and said hybridization results in formation of a third nick structure between a terminus of said nucleic acid analyte and a second terminus of said second oligonucleotide probe comprising said double-stranded terminal stem-loop structure, and said second terminus of said second oligonucleotide probe is part of said double-stranded terminal stem-loop structure of said second oligonucleotide probe.

3. The bioassay method according to claim 1, wherein either:
(a) said first terminuses are 5′ ends (five prime ends) and said second terminuses are 3′ ends (three prime ends), or
(b) said first terminuses are 3′ ends (three prime ends) and said second terminuses are 5′ ends (five prime ends).

4. The bioassay method according to claim 1, wherein said nucleic acid analyte is a single stranded nucleic acid with length of 10 - 50 nucleotides.

5. The bioassay method according to claim 1, wherein said nucleic acid analyte is microRNA (miRNA) with length of 17 - 25 nucleotides.

6. The bioassay method according to claim 1, wherein said first and second oligonucleotide probes comprise DNA (deoxyribonucleic acids) or RNA (ribonucleic acids) or any combination of them.

7. The bioassay method according to claim 1, wherein said first and second oligonucleotide probes are both labelled, or said first and second oligonucleotide probes are both labelled and, in addition, either first or second oligonucleotide probe is coupled to any kind of solid support.

8. The bioassay method according to claim 1, wherein said first and second oligonucleotide probes are labelled with a fluorescence resonance energy transfer pair, wherein, either:
(a) said first oligonucleotide probe comprises a fluorescent donor and said second oligonucleotide probe comprises a fluorescent acceptor or quencher, or
(b) said first oligonucleotide probe comprising a fluorescent acceptor or quencher and said second oligonucleotide probe comprising a fluorescent donor; and
wherein resonance energy transfer between said fluorescence resonance energy transfer pair is enabled upon occurrence of both said hybridization events, hybridization of said single-stranded terminal sequence overhang of said first oligonucleotide probe to said first region of the nucleic acid analyte, and hybridization of said single-stranded terminal sequence of said second oligonucleotide probe to said second region of the nucleic acid analyte.

9.The bioassay method according to claim 8,wherein said fluorescence resonance energy donor is a luminescent lanthanide chelate comprising a lanthanide ion.

10.The bioassay method according to claim 1,wherein said first and second oligonucleotide probes are labelled with a switchable lanthanide luminescence label system, wherein, either:
(a) said first oligonucleotide probe comprises a lanthanide ion carrier ligand and a lanthanide ion and said second oligonucleotide probe comprises an antenna ligand, or
(b) said first oligonucleotide probe comprises an antenna ligand and said second oligonucleotide probe comprises a lanthanide ion carrier ligand and a lanthanide ion; and wherein luminescence of said switchable lanthanide luminescence label system is switched on upon occurrence of both said hybridization events, hybridization of said single-stranded terminal sequence of said first oligonucleotide probe to said first region of the nucleic acid analyte, and
hybridization of said single-stranded terminal sequence of said second oligonucleotide probe to said second region of the nucleic acid analyte.

11.The bioassay method according to claim 9, wherein said lanthanide ion is selected from the group consisting of praseodymium(III), neodymium(III), samarium(III), europium(III), terbium(III), dysprosium(III), holmium(III), erbium(III), thulium(III) and ytterbium(III).

12.The bioassay method according to claim 2,wherein a ligase enzyme is used to form a covalent bond in a place of any one or any combination of said first, second and third nick structures formed upon occurrence of any of said hybridization events between said first oligonucleotide probe, said second oligonucleotide probe and the nucleic acid analyte.

13. A kit for detecting and/or quantitating a short nucleic acid analyte molecule, the kit comprising:
(a) a first oligonucleotide probe comprising a double-stranded terminal stem-loop structure and a single-stranded terminal sequence overhang that is complementary to and is capable of selectively hybridizing with a first region of a single-stranded nucleic acid analyte, and
b) a second oligonucleotide probe comprising a double-stranded terminal stem-loop structure and a single-stranded terminal sequence overhang that is complementary to and is capable of selectively hybridizing with a second region of said single-stranded nucleic acid analyte,
wherein said first and second region of the single-stranded nucleic acid analyte are strictly adjacent regions of said single-stranded nucleic acid analyte,
wherein a terminal nucleotide in a terminus of the single-stranded terminal sequence overhang of said first oligonucleotide probe and a terminal nucleotide in a terminus of the single-stranded terminal sequence overhang of said second oligonucleotide probe are designed to bind to adjacent nucleotides in said single-stranded nucleic acid analyte in order to form a first nick structure between said terminal nucleotides of the single-stranded terminal sequence overhangs of said first and second oligonucleotide probes when said probes are bound to said single-stranded nucleic acid analyte, said first nick structure lacking a phosphodiester bond between a pair of adjacent nucleotides bound to a complementary strand; and
wherein said single-stranded terminal sequence overhangs of said first and second oligonucleotide probes are further designed so that when said probes are bound to said single-stranded nucleic acid analyte, one of the terminal nucleotides of said single-stranded nucleic acid analyte forms a second nick structure with a terminal nucleotide in a terminus which is part of said double-stranded terminal stem-loop structure of the first oligonucleotide probe and another of the terminal nucleotides of said single-stranded nucleic acid analyte forms a third nick structure with a terminal nucleotide in a terminus which is part of said double-stranded terminal stem-loop structure of the second oligonucleotide probe, said second and third nick structures lacking a phosphodiester bond between a pair of adjacent nucleotides bound to a complementary strand.

14. The kit according to claim 13, wherein:
i) said first oligonucleotide probe comprises a 5′ end at the terminus of the single-stranded terminal sequence overhang and said second oligonucleotide probe comprises a 3′ end at the terminus of the single-stranded terminal sequence overhang; or alternatively
ii) said first oligonucleotide probe comprises a 3′ end at the terminus of the single-stranded terminal sequence overhang and said second oligonucleotide probe comprises a 5′ end at the terminus of the single-stranded terminal sequence overhang.

15. The kit according to claim 13, comprising said first and second oligonucleotide probes bound to said single-stranded nucleic acid analyte as a control sample.

16. The kit according to claim 13, wherein said single stranded nucleic acid analyte is with length of 10 - 50 nucleotides.

17. The kit according to claim 13, wherein said single stranded nucleic acid analyte is a microRNA (miRNA), with length of 17 - 25 nucleotides.

18. The kit according to claim 13, wherein said first and second oligonucleotide probes are both labelled, or said first and second oligonucleotide probes are both labelled and, in addition, either first or second oligonucleotide probe is coupled to any kind of solid support.

19. The kit according to claim 18 , wherein said first and second oligonucleotide probes are labelled with a fluorescence resonance energy transfer pair, wherein, either
(a) said first oligonucleotide probe comprises a fluorescent donor and said second oligonucleotide probe comprises a fluorescent acceptor or quencher, or
(b) said first oligonucleotide probe comprising a fluorescent acceptor or quencher and said second oligonucleotide probe comprising a fluorescent donor; and
wherein resonance energy transfer between said fluorescence resonance energy transfer pair is enabled upon occurrence of both said hybridization events, hybridization of said single-stranded terminal sequence overhang of said first oligonucleotide probe to said first region of the nucleic acid analyte, and hybridization of said single-stranded terminal sequence overhang of said second oligonucleotide probe to said second region of the nucleic acid analyte.
